(19) United States
(12) Patent Application Publication
(10) Pub. No.: US 2020/0030431 A1
(43) Pub. Date: Jan. 30, 2020

(54) VACCINES, COMPOSITIONS AND METHODS FOR USE THEREOF TO PREVENT OR REDUCE SEVERITY OF CHOLERA

(71) Applicant: THE CHILDREN'S MEDICAL CENTER CORPORATION, Boston, MA (US)

(72) Inventors: Paula Watnick, Newton, MA (US); Julie Szu Yu Liao, Boston, MA (US)

(73) Assignee: THE CHILDREN'S MEDICAL CENTER CORPORATION, Boston, MA (US)

(21) Appl. No.: 16/285,788

(22) Filed: Feb. 26, 2019

Related U.S. Application Data
(60) Provisional application No. 62/703,527, filed on Jul. 26, 2018.

(51) Int. Cl. A61K 39/02

(52) U.S. Cl. CPC A61K 39/107; A61K 2039/522

(57) ABSTRACT

Provided herein are methods and compositions comprising a Vibrio cholerae bacterium decorated with a fusion protein comprising an antigenic moiety. Such compositions have an increased antigenicity as compared to the same strain of Vibrio cholerae that is not decorated with the fusion protein.

### CROSS REFERENCE TO RELATED APPLICATIONS

This application claims benefit under 35 U.S.C. § 119(e) of U.S. Provisional Application 62/703,527 filed on Jul. 26, 2018, the contents of which is incorporated herein by reference in its entirety.

### FIELD OF THE INVENTION

The field of the invention relates to whole cell vaccine compositions and methods of use thereof in the prevention of cholera.

### BACKGROUND

Diarrhea is the most common infection in children under the age of five, worldwide. In spite of this, only a few vaccines to treat infectious diarrhea exist, and many of the available vaccines are sparingly and sporadically administered. Major obstacles to the development and widespread implementation of vaccination include the ease and cost of production, distribution, and delivery.

Diarrheal illness can be caused by e.g., Vibrio cholerae, Shigella spp., or enterotoxigenic Escherichia coli (ETEC).

### SUMMARY

Provided herein, in part, are improved vaccines for inducing immunity to e.g., Vibrio cholerae, which, unlike current vaccines, do not require large amounts of scarce potable water during administration. In particular, provided herein are methods and compositions comprising a Vibrio cholerae bacterium decorated with a fusion protein comprising an antigenic moiety. Such compositions have an increased antigenicity as compared to the same strain of Vibrio cholerae that is not decorated with the fusion protein.

It is further contemplated herein that these methods can be extended to the preparation of vaccines for other bacterial species that cause e.g., diarrheal illnesses. In certain embodiments, a strain of Vibrio cholerae “decorated” as described herein can be used with an antigen from another bacterial species, such as Shigella or enterotoxigenic E. coli. However, it may be desirable to decorate the bacterial species (e.g., Shigella or ETEC) with an antigen isolated or derived from the same bacterial species, thus increasing the number of epitopes for a given bacterial species and enhancing immunity against that same species. For example, ETEC bacteria can be decorated with a fusion protein comprising a heat-stable toxoid (Sta^{A14H}), a non-toxigenic form of the heat-stable toxin secreted by the ETEC bacteria, thereby enhancing the efficacy of a vaccine to ETEC-derived illness. One of skill in the art can identify a moiety that binds an extracellular polysaccharide on the surface of e.g., ETEC bacteria, analogous to RbmA for Vibrio cholerae, for generating a fusion protein as described herein.

Provided herein are compositions comprising a decorated bacterium (e.g., Vibrio cholerae, Shigella spp., or enterotoxigenic E. coli) having a fusion protein associated with its extracellular surface, wherein the fusion protein comprises a moiety that binds an extracellular polysaccharide on the bacterium and an antigenic moiety.

Accordingly, in one aspect, provided herein is a composition comprising a decorated Vibrio cholerae bacterium having a fusion protein associated with its extracellular surface, wherein the fusion protein comprises a moiety that binds Vibrio polysaccharide (VPS) and an antigenic moiety.

In one embodiment of this aspect and all other aspects provided herein, the Vibrio cholerae bacterium is inactivated or live-attenuated.

In another embodiment of this aspect and all other aspects provided herein, the immunogenicity of the decorated bacterium is increased by at least 10% compared to the same bacterium in the absence of the fusion protein.

In another embodiment of this aspect and all other aspects provided herein, the fusion protein is non-covalently attached to the surface of the bacterium.

In another embodiment of this aspect and all other aspects provided herein, the moiety that binds VPS is a lectin.

In another embodiment of this aspect and all other aspects provided herein, the lectin is RbmA or a C-terminal portion thereof. In certain embodiments, the RbmA comprises at least one mutation. In another embodiment, the lectin is RbmA with a point mutation, such as R116A and/or R234A.

In another embodiment of this aspect and all other aspects provided herein, the antigenic moiety comprises B subunit of cholera toxin (CTB), an enterotoxigenic E. coli (ETEC) antigen or a Shigella antigen. In one embodiment, the ETEC antigen is a toxoid (e.g., an inactive, immunogenic variant of “heat stable protein” such as STa or “heat-labile protein” such as LT in reference to antigens from ETEC). In one embodiment, the antigenic moiety isolated or derived from ETEC comprises the heat-stable enterotoxin STa. In another embodiment, the Shigella antigen comprises the outer membrane protein OmpC or a portion of this protein or a component of the type 3 secretion system of Shigella, for example, IpaB or the host cell invasion protein IpaD or portions of these proteins.

In another embodiment of this aspect and all other aspects provided herein, the fusion protein is expressed from a plasmid by the bacterium. Alternatively, the fusion protein is expressed from the bacterial chromosome.

In another embodiment of this aspect and all other aspects provided herein, expression of the fusion protein is under the control of an inducible promoter.

In another embodiment of this aspect and all other aspects provided herein, expression of the fusion protein is under the control of a chromosomal promoter.

In another embodiment of this aspect and all other aspects provided herein, the plasmid or engineered bacterial chromosome further encodes at least one additional antigen. In certain embodiments, the at least one additional antigen is operably linked to a chromosomal promoter.

In another embodiment of this aspect and all other aspects provided herein, the at least one additional antigen comprises multiply-mutated cholera toxin (mmCT).

In another embodiment of this aspect and all other aspects provided herein, the composition is formulated as a whole cell vaccine.

In another embodiment of this aspect and all other aspects provided herein, the composition is formulated for sublingual delivery.

In another embodiment of this aspect and all other aspects provided herein, the composition further comprises an adjuvant.

In another embodiment of this aspect and all other aspects provided herein, the adjuvant is mmCT.

Another aspect provided herein relates to a method for inducing immunity to Vibrio cholerae in a subject, the method comprising: administering at least one dose of the composition of claim 10 to a subject at risk of being exposed to and/or developing cholera, thereby inducing immunity to Vibrio cholerae in the subject.

In one embodiment of this aspect and all other aspects provided herein, the dose is a single dose comprising an effective amount of the composition.

In another embodiment of this aspect and all other aspects provided herein, the dose is administered at least twice.

In another embodiment of this aspect and all other aspects provided herein, the second dose is administered as a booster.

In another embodiment of this aspect and all other aspects provided herein, each dose comprises an amount of the composition that does not induce immunity alone and wherein the cumulative effect of at least two doses of the composition results in immunity to Vibrio cholerae.

In another embodiment of this aspect and all other aspects provided herein, the administering does not require sodium bicarbonate in potable water.

### BRIEF DESCRIPTION OF THE FIGURES

FIGS. 1A-1F. Generation of a prototype biofilm matrix protein vaccine. (FIG. 1A) Schematic demonstrating the use of RbmA to anchor the B subunit of cholera toxin (CTB), a secreted protein, to the surface of the cell by fusion to the C terminus of RbmA. (FIG. 1B) Genotype of strains expressing cholera toxin B subunit (CTB) from the tac promoter on a multi-copy plasmid either alone (pCTB) or genetically coupled to the 3′ end of rbmA (pR-CTB). MO10(pR-CTB) constitutes a prototype antigen-boosted whole cell vaccine. (FIG. 1C) Western blot analysis of cells harboring an empty expression plasmid (EV) or the same plasmid expressing either CTB or the RbmA-CTB fusion protein (R-CTB). A polyclonal primary antibody against CTB was used. The molecular masses of purified, monomeric CTB and the RbmA-CTB fusion protein are 11.6 kDa and 42.7 kDa, respectively (arrows). (FIG. 1D) Quantification of R-CTB per cell in a Vc(pR-CTB) prototype whole cell vaccine or a Dukoral® (WC-rBS) equivalent. (FIG. 1E) Western analysis showing that R-CTB is released into the supernatant by a V. cholerae ΔvpsA mutant. The pellet and supernatants of three independent cultures representing biological triplicates are shown for each condition. (FIG. 1F) Quantification of integrated band intensities in (FIG. 1E) by densitometry. The mean measurement is shown. Error bars represent the standard deviation. A one-way ANOVA was used to calculate statistical significance. **p<0.01

FIGS. 2A-2C. A prototype inactivated vaccine administered via the orogastric route does not elicit CTB-specific antibody responses. (FIG. 2A) Western blot analysis of CTB and R-CTB cell association after formalin treatment of a prototype vaccine. R-CTB (arrow), but not native CTB, remains in the cellular fraction after formalin treatment. Formalin-treatment results in crosslinking of R-CTB. (FIG. 2B) Vaccination and sample collection time line. Open triangles indicate vaccination. Red arrows indicate blood and stool collection. (FIG. 2C) Fold change of CTB-specific IgA and IgG in the serum and CTB-specific IgA in the stool. Antibody levels were measured four weeks after the second vaccine booster. Fold-change was calculated using the antibody response of PBS-immunized mice as the denominator. Each vaccination group included ten mice. Horizontal bars mark the mean. **p<0.01, n.s. not significant using one-way ANOVA followed by Tukey's test.

FIGS. 3A-3I. A sublingually-delivered, live-attenuated, whole cell vaccine expressing R-CTB elicits LPS and CTB-specific serum and stool antibodies and is protective in an infant mouse model of cholera. (FIG. 3A) Genotype of the live-attenuated V. cholerae strain harboring pR-CTB that was used for vaccination. (FIG. 3B) Vaccination and sample collection time line. Open triangles indicate vaccination. Red arrows indicate blood and stool collection. (FIG. 3C) Live V. cholerae recovered from stool pellets after sublingual immunization. Bacterial shedding ceased after 24 hours. The limit of detection is 440 CFU/g stool and is denoted by the dotted line. Data for groups receiving the vaccine strain alone or expressing R-CTB are shown. Each group included ten mice. (FIG. 3D) Fold change of LPS-specific IgG and IgA in the serum and LPS-specific IgA in the stool of mice immunized with the sublingual, live-attenuated vaccine. Each vaccination group included ten mice. (FIG. 3E) Comparison of serum vibriocidal titers at day 42 post immunization with the sublingual, live-attenuated vaccine or the formalin-inactivated orogastrically-administered vaccine. (FIG. 3F) Colonization of the small and large intestines of suckling mice challenged with wild-type MO10. Pups were born to unvaccinated dams (Ctrl) or to dams that received the live-attenuated, sublingual vaccine (Vacc). Litters arising from vaccinated and unvaccinated dams included 16 and 11 pups, respectively. Representative (FIG. 3G) intestinal fluid accumulation in the large intestine and cecum (triangle) and (FIG. 3H) skin turgor of pups in vaccinated (Vacc) or control (Ctrl) litters, respectively. Scale bar=1 cm. (FIG. 3I) Fold change of CTB-specific IgG and IgA in the serum and CTB-specific IgA in the stool of mice immunized with a control vaccine strain or a vaccine strain expressing R-CTB. Each vaccination group included ten mice. Horizontal bars mark the median in FIGS. 3C, 3E, and 3F. Horizontal bars in FIGS. 3D and 3I mark the mean. ns, not significant, *p<0.5, **p≤0.01, ***p≤0.001, ****p≤0.0001 using one-way ANOVA followed by Tukey's test in FIGS. 3D and 3I. A complete set of statistical comparisons is given in Table 2. Two-tailed, unpaired Mann-Whitney U-test was used in FIGS. 3C, 3E, and 3F.

FIG. 4. Total protein loaded for Western blot analysis of R-CTB in wild-type and ΔvpsA strains. Total protein in the pellet fractions of wild-type and ΔvpsA strains were comparable between each strain and between biological triplicates. R-CTB has a predicted molecular mass of 42.8 kD.

FIGS. 5A-5F. Chromosomally expressed RΔ-CTB is poorly immunogenic. (FIG. 5A) Genotype of an O139 serotype vaccine strain in which the entire CTX phage, including the recombination sites, as well as the tcpA gene has been deleted. Two R→A mutations (R116A, R234A) were introduced into rbmA. CTB is genetically coupled to the 3′ end of rbmA and placed under the native rbmA promoter on the chromosome (cRΔ-CTB). (FIG. 5B) Western blot analysis of CTB fused to the C-terminal end of wild-type RbmA (WT) or an RbmA protein carrying the R116A and R234A point mutations was detected in the V. cholerae cell pellet. (FIG. 5C) Quantification of cell-associated CTB in femtogram (fg) per cell when expressed from a plasmid or on the chromosome. Error bars in denote standard deviation, *p<0.05 using two-tailed, unpaired Student's t test. (FIG. 5D) Vaccination scheme for the live-attenuated vaccines. Open triangles indicate vaccination. Arrows indicate blood and stool sample collection. (FIG. 5E) Fold change of LPS-specific antibodies after immunization with a live-attenuated vaccine strain expressing cRΔ-CTB. *p<0.05 and ****p<0.0001 using one-way ANOVA followed by Dunnett's test for multiple comparison. (FIG. 5F) Fold change of CTB-specific antibodies after immunization with a live-attenuated vaccine strain expressing cRΔ-CTB. *p≤0.05 and ****p≤0.0001 using one-way ANOVA followed by Dunnett's test for multiple comparison. Horizontal bars mark the mean. Each vaccination group included ten mice. Fold change calculated against pre-immune background levels.

FIGS. 6A-6E. Characterization of mmCT as an in vivo antigen and adjuvant. (FIG. 6A) Genotype of an O139 serotype vaccine strain in which the entire CTX phage, including the recombination sites, as well as the tcpA gene has been deleted. The genes encoding mmCT, a cholera toxin variant carrying multiple mutations in the A subunit of cholera toxin, are integrated in-frame into the lacZ gene on the V. cholerae chromosome. A ribosome-binding site is included at the 5′ end of the sequencing encoding mmCT, allowing the lacZ promoter to drive transcription and translation of mmCT. (FIG. 6B) Constitutive production of mmCT in LB medium does not affect bacterial fitness as measured by growth over time. (FIG. 6C) Western blot analysis of cell pellets and supernatants after overnight LB broth culture of the vaccine strain noted in FIG. 6A with or without mmCT. Rabbit sera raised against CT followed by secondary anti-rabbit antibody were used for detection. (FIG. 6D) Quantification of mmCT in the supernatant of an overnight culture in LB and after incubation of the vaccine preparation in PBS. Vaccines were kept on ice for less than 1 hr before administration. (FIG. 6E) Quantification of the amount of CTB delivered as R-CTB and as part of mmCT, compared to the amount of purified CTB in Dukoral®. The amount of CTB from supernatant mmCT was measured after 1 hr of incubation at 22° C. in PBS.

FIGS. 7A-7D. In situ production of mmCT alone after sublingual vaccine delivery elicits a robust and long-lived immune response to CTB. (FIG. 7A) Genotype of vaccine strain used. Constitutive expression of genes encoding mmCT is driven by the constitutive lacZ promoter on the chromosome of the vaccine strain. (FIG. 7B) Vaccination scheme for the live-attenuated vaccines with or without constitutive expression of mmCT. Open triangles indicate vaccination. Arrows indicate blood and stool sample collection. (FIG. 7C) Fold change of LPS-specific antibodies after immunization with a live-attenuated vaccine strain expressing mmCT. (FIG. 7D) Fold change of CTB-specific antibodies after immunization with a live-attenuated vaccine strain expressing mmCT. * p≤0.05, **p≤0.01, ***p≤0.001, ****p≤0.0001, using one-way ANOVA and Dunnett's test. Horizontal bars mark the mean. Each vaccination group included ten mice. Fold change calculated against pre-immune background levels.

FIGS. 8A-8D. Co-expression of the adjuvant mmCT with chromosomal RΔ-CTB attenuates the immune response to CTB. (FIG. 8A) Genotype of an O139 serotype vaccine strain in which the entire CTX phage, including the recombination sites, as well as the tcpA gene has been deleted. Constitutive expression of genes encoding mmCT is driven by the native, chromosomal lacZ promoter. CTB is genetically coupled to the 3′ end of rbmA carrying R116A, R234A mutations and placed under the native rbmA promoter on the chromosome (cRΔ-CTB). (FIG. 8B) Vaccination scheme for the live-attenuated vaccine with chromosomal RΔ-CTB and constitutive expression of mmCT. Open triangles indicate vaccination. Arrows indicate blood and stool sample collection. Fold change of (FIG. 8C) LPS-specific and (FIG. 8D) CTB-specific antibodies after immunization with a live-attenuated vaccine strain expressing cRΔ-CTB and mmCT. n.s. p≥0.05, *p≤0.05, **p≤0.01, ***p≤0.001, ****p≤0.0001 using one-way ANOVA and Dunnett's multiple comparison test. Horizontal bars mark the mean. Each vaccination group included ten mice. Fold change calculated against pre-immune background levels.

FIGS. 9A-9D. Higher expression of R-CTB along with mmCT further dampens the immune response to CTB. (FIG. 9A) Genotype of an O139 serotype vaccine strain in which the gene encoding the A subunit of cholera toxin (ctxA) is deleted. Constitutive expression of genes encoding mmCT is driven by the native, chromosomal lacZ promoter. CTB genetically coupled to the 3′ end of wild-type rbmA is placed under a P_{tac} promoter on a multicopy plasmid (pR-CTB). (FIG. 9B) Vaccination scheme for the live-attenuated vaccine with plasmid-encoded pR-CTB and constitutive expression of mmCT. Open triangles indicate vaccination. Arrows indicate blood and stool sample collection. Fold change of (FIG. 9C) LPS-specific and (FIG. 9D) CTB-specific antibodies after immunization with a live-attenuated vaccine strain expressing plasmid-encoded pR-CTB and chromosomally-encoded mmCT. n.s. p≥0.05, ***p≤0.001, ****p≤0.0001 using one-way ANOVA and Dunnett's multiple comparison test. Horizontal bars mark the mean. Each vaccination group included ten mice. Fold change calculated against pre-immune background levels.

FIG. 10. Total protein loaded for Western blot analysis of R-CTB in wild-type and Δvps strains. Total protein in the pellet fractions of wild-type and ΔvpsA strains were comparable between each strain and between biological triplicates. R-CTB has a predicted molecular mass of 42.8 kDa.

FIG. 11. Dissemination of live bacteria lacking the essential colonization factor TCP following sublingual immunization occurs at low levels and is not significantly affected by presence of the adjufant mmCT. Live V. cholerae recovered from stool pellets after sublingual immunization with live-attenuated vaccine strains harboring a deletion in the gene encoding the major subunit of the toxin co-regulated pilus, which is necessary for in vivo colonization. Bacterial shedding ceased after 24 hours. The limit of detection is 440 CFU/g stool and denoted by the dotted line. Each vaccination group included 10 mice. Horizon bars mark the median. ns, not significant using two-tailed, unpaired Mann Whitney U-test.

FIGS. 12A-12E. Heterologous protein antigens incorporated into a live-attenuated, sublingual vaccine stimulate production of antigen-specific systemic and mucosal antibodies. (FIG. 12A) Genotype of vaccine strain used. LTB and an immunogenic, detoxified form of STa (STa^{A14H}), are fused to the C-terminus of RbmA for presentation on the cell surface. (FIG. 12B) Western blot analysis of pellets and supernatants after overnight culture of vaccine strains that do or do not express R-LTB-STa (R-LT/ST). An STa-specific antibody was used. The RbmA-LTB-STa fusion protein has a calculated molecular mass of 45.7 kDa and is indicated by the arrow. (FIG. 12C) Vaccination scheme for the live-attenuated vaccines that express heterologous antigens from ETEC with or without co-expression of mmCT. Open triangles indicate vaccination. Arrows indicate blood and stool collection. Fold change of (FIG. 12D) LTB/CTB-specific and (FIG. 12E) STa-specific antibodies after immunization with a vaccine expressing R-LT/ST with or without co-expression of mmCT. n.s. p≥0.05, *p≤0.05, **p≤0.01, ***p≤0.001, ****p≤0.0001, one-way ANOVA and Tukey's multiple comparison test. Horizontal bars mark the mean. Each vaccination group included ten mice. Fold change calculated against pre-immune background levels.

### DETAILED DESCRIPTION

The compositions and methods described herein are related, in part, to the discovery that the degree of antigenicity of a whole cell vaccine comprising Vibrio cholerae can be increased by the addition of an extracellular antigen bound to the bacterium's surface. Thus, vaccines with increased effectiveness can be generated because they may provide additional epitopes for the immune system to generate an antibody against, or have increased adjuvanticity. Extracellular antigens can be isolated from a variety of different pathogenic organisms to be attached to the exterior surface of Vibrio cholerae to form a whole cell vaccine against a desired antigen. It is also contemplated herein that other pathogenic bacteria, such as Shigella or Enterotoxigenic E. coli (ETEC) can be decorated as described herein with antigens from the same or different bacteria attached to their surface to enhance an immune response to a vaccine against shigellosis or ETEC-derived diarrheal disease.

### Definitions

The term “vaccine” is used herein to define a composition used to elicit an immune response against an antigen within the composition in order to protect or treat an organism against disease.

As used herein, the term “antigen” refers to any substance that prompts an immune response directed against the substance. In some embodiments, an antigen is a peptide, a polypeptide or a displayed polypeptide.

As used herein, the term “fused” means that at least one protein, peptide, or polypeptide is physically associated with a second protein or peptide, such as linkage as a fusion protein.

As used herein, the term “fusion protein” or “antigenic fusion protein” means a protein created by joining two or more polypeptide sequences together. The fusion polypeptides encompassed in the methods and compositions described herein can include e.g., a protein that binds Vibrio polysaccharide (VPS) and an antigenic moiety. In other words, a “fusion polypeptide” or “fusion protein” is a recombinant protein of two or more proteins (or fragments thereof) which are joined by a peptide bond.

As used herein, the term “decorated bacterium” or “functionalized bacterium” refers to a bacterium (e.g., Vibrio cholerae) that comprises an exogenous fusion protein associated with the outer membrane cell surface of the bacterium. The fusion protein can be non-covalently associated, for example, by interaction of a lectin moiety in the fusion protein with the polysaccharides on the cell's surface (e.g., VPS) or can be covalently attached, if desired. For the purposes of the methods and compositions provided herein, the fusion protein comprises an antigenic moiety such that the immunogenicity of the bacterium is increased upon decoration (i.e., attachment or association) of its cell surface as compared to immunogenicity of a substantially similar, but undecorated, bacterium. In one embodiment, the decorated bacterium is Vibrio cholerae.

As used herein, the terms “more immunogenic,” “increased antigenicity” or “increased immunogenicity” each refer to the increased ability of a decorated bacterium as described herein to invoke an immune response compared to the same bacterial strain that is not decorated as described herein. In some embodiments, the ability of the composition comprising a decorated bacterium to invoke an immune response can be determined by measuring the amount of antibody production or titer in a cell culture or animal model and comparing that to the amount of antibody production or antibody titer in response to an undecorated bacterium using the same model. At a minimum, a composition comprising a decorated bacterium is more immunogenic if the antibody titer is at least 10% higher in such an assay. In some embodiments, the immunogenicity of the composition as described herein is determined by measuring the activation of immune effector cells. Methods and assays for determining the immunogenicity of the composition comprising a decorated bacterium are described herein in the section marked “Measuring Immunogenicity.” Typically, a decorated bacterium has an increased immunogenicity of at least 10% compared to the measured indicator of immunogenicity produced by the same bacterial strain but lacking the fusion protein on the cell surface; preferably the decorated bacterium produces an increase in immunogenicity of at least 20%, at least 30%, at least 40%, at least 50%, at least 60%, at least 70%, at least 80%, at least 90%, at least 95%, at least 98%, at least 99%, at least 1-fold, at least 2-fold, at least 5-fold, at least 10-fold, at least 50-fold, at least 100-fold, at least 500-fold, at least 1000-fold (or more) higher than the immunogenicity observed in the same model in response to an undecorated bacterium.

The term “adjuvant” as used herein refers to any agent or entity which increases the antigenic response or immune response by a cell or organism to a target antigen. Examples of adjuvants include, but are not limited to mineral gels such as aluminum hydroxide or aluminum phosphate; surface active substances such as lysolecithin, pluronic polyols, polyanions; other peptides; oil emulsions; and potentially useful human adjuvants such as BCG and Corynebacterium parvum, QS-21, Detox-PC, MPL-SE, MoGM-CSF, TiterMax-G, CRL-1005, GERBU, TERamide, PSC97B, Adjumer, PG-026, GSK-I, GcMAF, B-alethine, MPC-026, Adjuvax, Albumin (Alum), CpG ODN, Betafectin, and MF59. In one embodiment, the adjuvant is not Freund's adjuvant (particularly for immunization of human subjects). In another embodiment, the adjuvant is a mucosal adjuvant (e.g., multiply mutated cholera toxin (mmCT).

The term “subject” as used herein refers to any animal in which it is useful to elicit an immune response. The subject can be a mammal, for example a human, or can be a wild, domestic, commercial or companion animal. While in one embodiment it is contemplated that the immunogenic compositions as disclosed herein can also be suitable for the therapeutic and/or preventative treatment in humans, it is also applicable to all vertebrates, e.g., mammals, such as non-human primates, (particularly higher primates), sheep, dog, rodent (e.g., mouse or rat), guinea pig, goat, pig, cat, rabbits, cows, and non-mammals such as chickens, amphibians, reptiles etc. In one embodiment, the subject is human. In some embodiments, the subject is an experimental animal or animal substitute as a disease model.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “prevention” or “preventing,” when used in reference to a disease, disorder or symptoms thereof, refers to a reduction in the likelihood that an individual will develop a disease or disorder, e.g., cholera, as but one example. The likelihood of developing a disease or disorder is reduced, for example, when an individual having one or more risk factors for a disease or disorder either fails to develop the disorder or develops such disease or disorder at a later time or with less severity, statistically speaking, relative to a population having the same risk factors and not receiving treatment as described herein. The failure to develop symptoms of a disease, or the development of reduced (e.g., by at least 10% on a clinically accepted scale for that disease or disorder) or delayed (e.g., by days, weeks, months or years) symptoms is considered effective prevention.

The terms “decrease”, “reduced”, “reduction”, or “inhibit” are all used herein to mean a decrease or lessening of a property, level, or other parameter by a statistically significant amount. In some embodiments, “reduce,” “reduction” or “decrease” or “inhibit” typically means a decrease by at least 10% as compared to a reference level and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level.

The terms “increased”, “increase” or “enhance” or “activate” are all used herein to generally mean an increase of a property, level, or other parameter by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, at least about a 20-fold increase, at least about a 50-fold increase, at least about a 100-fold increase, at least about a 1000-fold increase or more as compared to a reference level.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth.

### Cholera: Treatment and Prevention

Cholera is a severe and potentially lethal diarrheal disease caused by the Gram-negative bacterium Vibrio cholera. When ingested, the bacteria colonize the small intestine, where they release cholera toxin (CT). This toxin causes the secretion of large amounts of water into the intestine, resulting in the profuse watery diarrhea characteristic of cholera.

Cholera is the most severe of many diarrheal diseases that affect humans. It is unusual in the speed with which dehydration and death can occur. In severe cases, patients may develop hypovolemic shock and acidosis, and can die in as short a period as 24 hours. Mortality rates in untreated patients can reach 70%.

The long-term control of cholera requires good personal hygiene, access to an uncontaminated water supply and appropriate disposal of human waste. However, these elements are not available in many developing or war-torn countries. Thus, the availability of an effective cholera vaccine is important for the prevention of cholera.

Current vaccines for cholera include (i) a vaccine comprising inactivated bacterial cells and the B-subunit of cholera toxin, (ii) a live attenuated vaccine containing the genetically manipulated V. cholerae O1 strain CVD 103-HgR and (iii) conjugates of V. cholerae O1 lipopolysaccharide with cholera toxin variants prepared with an adipic acid dihydrazide linker.

### Other Gastrointestinal Dysentries

It is contemplated herein that the methods and compositions described herein regarding vaccines to Vibrio cholerae can be extended to the production of vaccines against bacterial dysentery caused by other pathogenic bacteria, such as Shigella spp., or enterotoxic E. coli. One of skill in the art can easily adapt the methods of decorating a Vibrio cholerae cell with a fusion protein as described herein to the generation of e.g., a decorated Shigella bacterium comprising an antigenic moiety that further increases the degree of immune response to a vaccine as compared to the degree of immune response that is generated by administration of Shigella bacterium alone.

Shigella: Shigellosis is a gastrointestinal illness caused by members of the Shigella family of bacteria including, but not limited to Shigella dysenteriae, Shigella flexneri, Shigella boydii, and Shigella sonnei. Shigella bacteria are invasive and penetrate the epithelial mucosa of the large intestine causing localized microvilli destruction. These bacteria can infect neighboring cells, resulting in the development of necrosis, acute inflammation, and epithelial ulcers. Infection by Shigella spp. is characterized by a small volume of painful, bloody diarrhea containing mucus. Treatment of shigellosis includes fluid replacement and rest.

There is no vaccine currently available for the successful vaccination against Shigella spp. and prevention of shigellosis. Prevention is thus limited to improvements in sanitation, hygiene or access to clean drinking water. In particular, a desirable vaccine to Shigella will not require administration using potable water, since clean drinking water may not always be available to those in need of vaccination.

Enterotoxigenic E. coli (ETEC); Infection with ETEC can cause severe diarrhea, dysentry, abdominal cramping and fever. This illness can be life-threatening if adequate fluids are not replaced, thus resulting in severe dehydration. ETEC bacteria colonize the mucosal surface of the small intestine and can secrete two different toxins (i.e., heat-labile (LT) and heat-stable (ST)), each of which stimulates intestinal cells to secrete fluid.

Primary means for reducing oral-fecal transmission of ETEC include building sanitation infrastructure, adequate cooking of food, peeling fruits/vegetables, and using boiled or chemically treated water.

Strains of Vibrio cholerae

Vibrio cholerae is a gram-negative bacterium that can be readily identified by its distinctive “comma” shape upon isolation from an infected subject. The pathogenicity of V. cholerae is due to a secreted toxin (cholera toxin) that causes watery diarrhea. An additional virulence factor allows the bacterium to colonize the small intestine by attachment of toxin coregulated pilus (TCP), a filamentous appendage on the surface of the cells to the mucosal lining. Widespread cholera transmission has been attributed to two serogroups of V. cholerae: strains O1 and O139.

It is specifically contemplated that any strain of Vibrio cholerae can be used to prepare the vaccine compositions as described herein. In addition, strains that have been inactivated or attenuated are also specifically contemplated for use herein.

In some embodiments, the strain of V. cholerae is of the O1 serogroup. Included in the O1 serogroup are strains of the El Tor and classical biotypes, both of which contain Inaba, Ogawa and Hikojima serotypes. For example, in some embodiments, the V. cholerae strain is E7946 (ATCC 55056), a clinical isolate from the 7th cholera pandemic belonging to the O1 serogroup, having the El Tor biotype, and having the Ogawa serotype.

In some embodiments, the strain of V. cholerae is of the O139 (Bengal) serogroup, implicated in outbreaks in Bangladesh and India in 1993.

In some embodiments, the strain of V. cholerae is of a different serogroup, such as O37, O22, O11, etc. In some embodiments, the strain of V. cholerae is of a yet to be identified serogroup that has emerged as a cause of one or more cholera outbreaks.

In certain embodiments, the strain of V. cholerae is a mutant version of a known isolate. The mutation may have occurred naturally (such as that might occur by errors in replication machinery, or as a result of environmental insults to the bacterial genome), or it may be introduced experimentally. For example, it may be desirable to introduce one or more mutations into a naturally occurring strain of V. cholerae such that the strain produces a heterologous gene product, such as mmCT, as described herein.

A list of strains of V. cholerae of O1, O139, and other serogroups, that can be used with the methods, vaccines and compositions described herein can be found in the UniProt database entry for V. cholerae (taxon identifier 666) available on the world wide web at beta.uniprot.org/taxonomy/666, herein incorporated by reference in its entirety.

### Antigenic Fusion Proteins

The fusion proteins described herein permit the decoration of the outer surface of a bacterium with an antigenic molecule, such that the decorated bacterium is more immunogenic than the bacterium alone. A fusion protein as described herein has two functional regions: (i) a moiety that permits binding or association of the fusion protein with the extracellular surface of the bacterium, and (ii) an antigenic peptide or portion thereof. In some embodiments, it may be necessary to modify or mutate the antigenic peptide or portion thereof, such that the antigenicity of the protein is retained but the adverse effects of the antigenic protein are reduced or eliminated. That is, at least one epitope for antibody generation is retained in a modified antigenic peptide or portion thereof. For example, the fusion proteins described herein can comprise the B subunit of the cholera toxin (CTB) or multiply mutated cholera toxin (mmCT).

It will be understood by one of skill in the art that it is most desirable to select an antigenic moiety for decorating a bacterium that will provide for antibody production and/or immunity to a disease caused by the same bacterial strain that is decorated. For example, provided herein are compositions comprising decorated Vibrio cholerae cells, which cause cholera and thus, the antigen chosen is a portion of the cholera toxin, thus permitting the production of antibodies to a variety of epitopes that are associated with cholera.

In one embodiment, the antigenic moiety is isolated from or derived from an enterotoxigenic E. coli (ETEC) or Shigella.

In one embodiment, the ETEC antigen comprises a heat stable toxoid (e.g., Sta) or heat-labile toxoid isolated or derived from ETEC.

Shigella spp. use a type-III secretion system to translocate toxic effector proteins to a target cell. Proteins involved in the secretion system are thus considered virulence factors. Such virulence factors isolated or derived from Shigella spp. and used as antigenic moieties include, but are not limited to, IpaA/IpaB, IpaC (C-terminal region), IpaC, VirA, ipaH, Osp's, and IpgB1. In another embodiment, the Shigella antigen comprises the translator protein IpaC or the host cell invasion protein IpaD.

Essentially any protein, peptide, carbohydrate, or ligand can be used as a moiety that permits association of the fusion protein to the extracellular surface of the bacterium. In one embodiment, such a moiety is a lectin or a portion thereof (e.g., the C terminus of RbmA).

Expression of the antigenic fusion protein can be under the control of an inducible promoter. Such promoters are known to those of skill in the art and, thus, are not described in detail herein.

Antigens can be isolated or derived from Campylobacter jejuni, Campylobacter coli, Listeria monocytogenes, Yersinia enterocolitica, Yersinia pestis, Yersinia pseudotuberculosis, Escherichia coli, Shigella flexneri, Shigella sonnei, Shigella dysenteriae, Shigella boydii, Helicobacter pylori, Helicobacter felis, Gastrospirillum hominus, Vibrio cholerae, Vibrio parahaemolyticus, Vibrio vulnificus, Bacteroides fragilis, Clostridium difficile, Salmonella typhimurium, Salmonella typhi, Salmonella gallinarum, Salmonella pullorum, Salmonella choleraesuis, Salmonella enteritidis, Klebsiella pneumoniae, Enterobacter cloacae, and Enterococcus faecalis. Escherichia coli include but are not limited to entero-toxic, entero-hemorrhagic, entero-invasive, entero-pathogenic or other strains. In certain embodiments, an antigen derived or isolated from such bacterial species comprises a secreted protein, a toxin or toxoid, or a virulence factor. In some embodiments, the antigen comprises at least one mutation.

In some embodiments, an antigenic moiety is selected that can provide immunity to more than one strain of e.g., Vibrio cholerae, Shigella spp., or enterotoxigenic E. coli. For example, an antigenic moiety can be selected that is conserved among the three Shigella flexneri serotypes and Shigella sonnei to provide a vaccine that induces immunity to multiple Shigella strains.

### Engineering Decorated Bacteria

In one embodiment, the methods and compositions described herein provide for the self-assembly of a decorated bacterium, which can in turn by used in the generation of a vaccine for inducing immunity of a subject to a given disease (e.g., cholera)

For self-assembly of a decorated bacterium, the bacterial strain is transformed with a plasmid comprising a nucleic acid encoding a fusion protein as described herein. In some embodiments, expression of the fusion protein is controlled by an inducible promoter as described herein. The fusion protein is secreted upon expression, and the VPS binding moiety associates with the cell surface polysaccharide, thereby self-assembling a decorated bacterium.

Essentially any pathogenic bacterium can be decorated as described for the purpose of generating a better vaccine. Some non-limiting examples of such bacteria include Campylobacter jejuni, Campylobacter coli, Listeria monocytogenes, Yersinia enterocolitica, Yersinia pestis, Yersinia pseudotuberculosis, Escherichia coli, Shigella flexneri, Shigella sonnei, Shigella dysenteriae, Shigella boydii, Helicobacter pylori, Helicobacter felis, Gastrospirillum hominus, Vibrio cholerae, Vibrio parahaemolyticus, Vibrio vulnificus, Bacteroides fragilis, Clostridium difficile, Salmonella typhimurium, Salmonella typhi, Salmonella gallinarum, Salmonella pullorum, Salmonella choleraesuis, Salmonella enteritidis, Klebsiella pneumoniae, Enterobacter cloacae, and Enterococcus faecalis. Escherichia coli include but are not limited to entero-toxic, entero-hemorrhagic, entero-invasive, entero-pathogenic or other strains.

In some embodiments, such pathogenic bacteria to be decorated are live, attenuated bacteria. In other embodiments, the pathogenic bacteria to be decorated are inactivated.

### Heterologous Gene Products

Provided herein are vaccine compositions comprising V. cholerae bacteria that comprise or express one or more heterologous gene products in addition to the fusion protein.

In some embodiments, a composition or vaccine as described herein can contain foreign proteins or other gene products not normally expressed by wild type strains of V. cholerae (e.g., mmCT). Genes encoding such proteins can be introduced into existing wild type or mutant strains of V. cholerae by methods known in the art, for example, using genetic elements such as transposable elements, viruses, plasmids, and etc. In some embodiments, such heterologous proteins are constitutively expressed by the bacterial strain after introduction of the foreign genetic element. Alternatively, the heterologous proteins are expressed by the bacterial strain after induction. For example, heterologous genes inserted into a plasmid and under the control of a lac repressor system may be introduced into V. cholerae. Inducers such IPTG (isopropyl beta-D-thiogalactoside) can be used to induce expression of proteins or gene products encoded by the heterologous genes.

In some embodiments, heterologous gene products are secreted and become associated or attached to the surfaces of the bacterium.

It is also contemplated herein that the heterologous gene product is derived from another bacterial species such as E. coli and expressed in V. cholerae bacteria. It may be desirable, for example, to express immunogenic gene products from other pathogens that infect via the gastrointestinal tract, such as Shigellae (such as, for example, Shigella dysenteriae, Shigella flexneri, Shigella boydii, Shigella sonnei), Salmonellae (such as, for example, S. enterica), Campylobacter jejuni, Helicobacter pylori, rotavirus, E. coli (including subtypes such as enterotoxigenic E. coli (ETEC), enteropathogenic E. coli (EPEC), enterohemorrhagic E. coli (EHEC), enteroinvasive E. coli (EIEC), Clostridium difficile and enteroaggregative E. coli (EAggEC), and/or combinations thereof. In one embodiment, an enterotoxigenic E. coli (ETEC) antigen or a Shigella antigen is expressed on the surface of Vibrio cholerae or a different pathogenic bacterium (e.g., ETEC, Shigella). In one embodiment, the ETEC antigen is a heat-stable toxoid (e.g., Sta). In another embodiment, the Shigella antigen comprises a component of the type 3 secretion system of Shigella, for example, the translator protein IpaC or the host cell invasion protein IpaD.

Vaccine compositions comprising such gene products may confer immunity to diseases mediated by such pathogens. Thus, in some embodiments, the compositions or vaccines described herein comprise gene products from more than one pathogenic species and may confer immunity to more than one infectious disease.

The heterologous gene product may be any kind of gene product whose expression can be achieved in V. cholerae. For example, the heterologous gene product may be cholera toxin B subunit. The gene may be constitutively expressed and/or inducible.

In some embodiments, the heterologous protein is a tag, such as a fluorescent protein. Such proteins can facilitate tracking and/or visualization of molecules. Examples of fluorescent proteins include, but are not limited to, green fluorescent protein (GFP) from the jellyfish Aequorea victoria; mutant versions of GFP that fluoresce different colors (such as BFP, blue fluorescent protein; YFP, yellow fluorescent protein; and CFP, cyan fluorescent protein); dsRed fluorescent protein (dsRed2FP); eqFP611, a red fluorescent protein isolated from Entacmaea quadricolor; AmCyan1, a cyan fluorescent protein isolated from Anemonia majano, and originally named amFP486; Azami Green, a bright fluorescent protein isolated from Galaxeidae; ZSGREEN™, a fluorescent protein isolated from Zoanthus; etc.

In some embodiments, the heterologous gene product is a gene product that enhances mucosal immunity. In other embodiments, the heterologous gene product comprises a secretion signal.

### Vaccine Formulation

The decorated bacteria (e.g., V. cholerae), as described herein, can be resuspended in a solution or buffer (such as, for example, sterile distilled water, saline, phosphate-buffered saline, etc.). In some embodiments, the compositions or vaccines contain no other components. In some embodiments, the compositions and vaccines described herein can comprise additional components to enhance stability, for example, protease inhibitor(s), osmolarity agents etc. Non-limiting examples of stabilizers include polyethylene glycol, proteins, saccharide, amino acids, inorganic acids, and organic acids which may be used either on their own or as admixtures. Two or more stabilizers can be used in aqueous solutions at the appropriate concentration and/or pH. The specific osmotic pressure in such aqueous solution is generally in the range of 0.1-3.0 osmoses, preferably in the range of 0.80-1.2. The pH of the aqueous solution is adjusted to be within the range of 5.0-9.0, preferably within the range of 6-8.

Alternatively, or additionally, adjuvants can be included in the compositions as described herein. Adjuvants may, in certain embodiments, enhance production of antibodies against V. cholerae and/or the antigenic fusion protein. Examples of suitable adjuvants include, but are not limited to, various oil formulations and/or emulsions such as stearyl tyrosine (see, for example, U.S. Pat. No. 4,258,029), muramyl dipeptide (also known as MDP, Ac-Mur-L-Ala-D), saponin, aluminum hydroxide, lymphatic cytokine, QS-21, Detox-PC, MPL-SE, MoGM-CSF, TiterMax-G, CRL-1005, GERBU, TERamide, PSC97B, Adjumer, PG-026, GSK-I, GcMAF, B-alethine, MPC-026, Adjuvax, CpG ODN, Betafectin, Alum, and MF59 etc. Adjuvants that are particularly suitable for inducing mucosal immunity include, but are not limited to, cholera toxin B subunit, heat labile enterotoxin (KT) from E. coli, Emulsomes (Pharoms, LTF., Rehovot, Israel), CpG oligodeoxynucleotides (ODNs), Toll-like receptor agonists, polyethyleneimine, chitosan, etc. In some embodiments, multiply mutated form of cholera toxin is used as an adjuvant.

In one embodiment, the composition or vaccine further comprises a delivery enhancer, for example, to increase penetration of the mucosal layer (e.g., polyethyleneimine, chitosan etc).

The compositions and vaccines described herein can be formulated for multiple administrations/immunizations, and an effective dose can be achieved by the administration of multiple immunizations whether or not each individual immunization comprises an effective dose.

In some embodiments, the compositions or vaccines are stored in a sealed vial, ampule, or similar container.

In some embodiments, the composition or vaccine is provided in a lyophilized form, which can improve ease in transportation and storage. In some such embodiments, vaccines are dissolved or suspended in a solution or buffer before administration.

In some embodiments, the composition or vaccine further comprises additional therapeutic agents (such as other vaccines or antigens associated with other diseases). In some embodiments, the other therapeutic agents do not diminish effectiveness of the vaccine composition for inducing immunity against cholera. In some embodiments, the vaccine or composition is administered in combination with other therapeutic ingredients including, e.g., interferons, cytokines, or chemotherapeutic agents. In some embodiments, the vaccine composition as disclosed herein can be administered with one or more co-stimulatory molecules and/or adjuvants as disclosed herein.

In one embodiment, the composition or vaccine as described herein comprises pharmaceutically acceptable carriers that are inherently nontoxic and nontherapeutic. Examples of such carriers include ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, and polyethylene glycol. For all administrations, conventional depot forms are suitably used. Such forms include, for example, microcapsules, nano-capsules, liposomes, plasters, inhalation forms, nose sprays, sublingual tablets, and sustained release preparations. For examples of sustained release compositions, see U.S. Pat. Nos. 3,773,919, 3,887,699, EP 58,481A, EP 158,277A, Canadian Patent No. 1176565; U. Sidman et al., Biopolymers 22:547 (1983) and R. Langer et al., Chem. Tech. 12:98 (1982).

In one embodiment, other ingredients can be added to vaccine formulations, including antioxidants, e.g., ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, mannose, or dextrins; chelating agents such as EDTA; and sugar alcohols such as mannitol or sorbitol.

The compositions and vaccines described herein can be formulated for any of a variety of routes of administration as discussed further below. For example, the compositions or vaccines can be formulated as a spray for intranasal inhalation, nose drops, swabs for tonsils, etc. The compositions or vaccines can be formulated for oral delivery in the form of capsules, tablets, gels, thin films, liquid suspensions and/or elixirs, etc. In one embodiment, the composition or vaccine is formulated for sublingual administration.

In some embodiments, the immunogenic compositions as described herein can be administered intravenously, intranasally, intramuscularly, subcutaneously, intraperitoneally, sublingually, vaginal, rectal or orally. In some embodiments, the route of administration is oral, intranasal, subcutaneous, or intramuscular. In some embodiments, the route of administration is sublingual, nasal, or oral administration.

When oral preparations are desired, the vaccine compositions can be combined with typical carriers, such as lactose, sucrose, starch, talc magnesium stearate, crystalline cellulose, methyl cellulose, carboxymethyl cellulose, glycerin, sodium alginate or gum arabic among others.

For some formulations (i.e., i.v. injection), the immunogenic compositions as described herein for administration must be sterile. Sterility is readily accomplished by filtration through sterile filtration membranes, or by gamma radiation.

In some embodiments, the vaccine composition is administered in a pure or substantially pure form, but it is preferable to present it as a pharmaceutical composition, formulation or preparation. Such formulation comprises decorated bacteria as described herein together with one or more pharmaceutically acceptable carriers and optionally other therapeutic ingredients.

### Dosage, Administration and Efficacy

As will be appreciated by one of skill in the art, appropriate dosing regimens for a given composition or vaccine can comprise a single administration/immunization or multiple ones. For example, vaccines can be given as a primary immunization followed by one or more boosters. Boosters may be delivered via the same and/or different route as the primary immunization. Boosters are generally administered after a time period following the primary immunization or the previously administered booster. For example, a booster can be given about two weeks or more after a primary immunization, and/or a second booster can be given about two weeks or more after the first boosters. Boosters may be given repeatedly at time periods, for example, about two weeks or greater throughout up through the entirety of a subject's life. Boosters can be spaced, for example, about two weeks, about three weeks, about four weeks, about one month, about two months, about three months, about four months, about five months, about six months, about seven months, about eight months, about nine months, about ten months, about eleven months, about one year, about one and a half years, about two years, about two and a half years, about three years, about three and a half years, about four years, about four and a half years, about five years, about ten years, about 20 years, about 30 years or more after a primary immunization or after a previous booster.

The precise dose to be employed in the formulation will also depend on the route of administration and should be decided according to the judgment of the practitioner and each patient's circumstances. Ultimately, the attending physician will decide the amount of protein or vaccine composition to administer to particular individuals. Doses generally each comprise between about 1×10⁶ and 1×10¹⁰ cells or more, for example, at least 5×10⁶, at least 1×10⁷, at least 5×10⁷, at least 1×10⁸, at least 5×10⁸, at least 1×10⁹, at least 5×10⁹, at least 1×10¹⁰, at least 5×10¹⁰ or more.

Vaccination can be conducted by conventional methods. For example, a displayed polypeptide can be used in a suitable diluent such as saline or water, or complete or incomplete adjuvants. The vaccine can be administered by any route appropriate for eliciting an immune response (e.g., sublingual, nasal, oral or intramuscular injection). The vaccine can be administered once or at periodic intervals until an immune response is elicited. Immune responses can be detected by a variety of methods known to those skilled in the art, including but not limited to, antibody production, cytotoxicity assay, proliferation assay and cytokine release assays. For example, samples of blood can be drawn from the immunized mammal, and analyzed for the presence of antibodies against the antigens of the immunogenic composition by ELISA (see de Boer G F, et. al., 1990, Arch Virol. 115:47-61) and the titer of these antibodies can be determined by methods known in the art.

In one embodiment, efficacy is determined by measuring the immunogenicity of the administered composition or vaccine, for example, by assessing immunity to the individual to which the composition is administered or immunity conferred to one or more offspring of the individual to which the composition is administered. For example, the individual being administered the composition can be a pregnant female, whose future or current offspring benefit from immune protection. Such immunity can be passed from mother to child, for example, through breastmilk and/or through blood exchanged between from mother and fetus via the placenta.

In some embodiments, antibody titer can be used as a measure of the humoral immunogenicity of a given composition or vaccine. As used herein, antibody titer is a measurement of how much antibody an organism, such as, for example, a human, a mouse or a rabbit, has produced that recognizes a particular epitope, expressed as the greatest dilution that still gives a positive result. ELISA is a common means of determining antibody titers, but other assays known to one of skill in the art can be used as well.

In other embodiments, a composition or vaccine as described herein elicits increased T cell responses or cell-mediated immunogenicity relative to the T cell immune response or cell-mediated immune response elicited when an undecorated bacterium is administered.

In other embodiments, efficacy can be determined by assessing a variety of clinical measures including, but not limited to, fewer cases of cholera than expected in a given population, a reduction in mortality, a reduction in the severity of cholera, reduction in diarrhea, reduction of dehydration, reduced number of hospitalizations etc.

The efficacy of a given composition for inducing immunity to cholera can be determined by the skilled clinician. However, a composition is considered “effective,” as the term is used herein, if any one or all of the signs or symptoms of the disease (e.g., cholera) is/are altered in a beneficial manner, or other clinically accepted symptoms or markers of disease are improved, or ameliorated, e.g., by at least 10% in individuals administered the composition compared to a substantially similar individual that has not been administered or immunized as described herein. Efficacy can also be measured by failure of an individual to worsen as assessed by stabilization of the disease (e.g., shorter duration, less intense symptoms), or the need for medical interventions (i.e., progression of the disease is halted or at least slowed). Methods of measuring these indicators are known to those of skill in the art and/or described herein.

In some embodiments, the subject is further evaluated using one or more additional diagnostic procedures, for example, by medical imaging, physical exam, laboratory test(s), clinical history, family history, gene tests, and the like. Medical imaging is well known in the art. As such, the medical imaging can be selected from any known method of imaging, including, but not limited to, ultrasound, computed tomography scan, positron emission tomography, photon emission computerized tomography, and magnetic resonance imaging.

The present invention may be as described in any one of the following numbered paragraphs:

1. A composition comprising a decorated Vibrio cholerae bacterium having a fusion protein associated with its extracellular surface, wherein the fusion protein comprises a moiety that binds Vibrio polysaccharide (VPS) and an antigenic moiety.

2. The composition of paragraph 1, wherein the Vibrio cholerae bacterium is inactivated or live-attenuated.

3. The composition of paragraph 1, wherein the immunogenicity of the decorated bacterium is increased by at least 10% compared to the same bacterium in the absence of the fusion protein.

4. The composition of paragraph 1, wherein the fusion protein is non-covalently attached to the surface of the bacterium.

5. The composition of paragraph 1, wherein the moiety that binds VPS is a lectin.

6. The composition of paragraph 5, wherein the lectin is RbmA or a C-terminal portion thereof.

7. The composition of paragraph 5, wherein the lectin is RbmA with a point mutation.

8. The composition of paragraph 7, wherein the lectin RbmA point mutation is R116A and/or R234A.

9. The composition of paragraph 1, wherein the antigenic moiety comprises B subunit of cholera toxin (CTB), a Shigella antigen or a heat-stable toxoid from enterotoxic E. coli.

10. The composition of paragraph 1, wherein the fusion protein is expressed from a plasmid by the bacterium.

11. The composition of paragraph 1, wherein expression of the fusion protein is under the control of an inducible promoter.

12. The composition of paragraph 1, wherein expression of the fusion protein is under the control of a chromosomal promoter.

13. The composition of paragraph 10, wherein at least one additional antigen is chromosomally expressed.

14. The composition of paragraph 13, wherein the at least one additional antigen comprises multiply-mutated cholera toxin (mmCT).

15. The composition of paragraph 1, wherein the composition is formulated as a whole cell vaccine.

16. The composition of paragraph 1, wherein the composition is formulated for sublingual delivery.

17. The composition of paragraph 1, further comprising an adjuvant.

18. The composition of paragraph 17, wherein the adjuvant is mmCT.

19. A method for inducing immunity to Vibrio cholerae in a subject, the method comprising: administering at least one dose of the composition of paragraph 1 to a subject at risk of being exposed to and/or developing cholera, thereby inducing immunity to Vibrio cholerae in the subject.

20. The method of paragraph 19, wherein the dose is a single dose comprising an effective amount of the composition.

21. The method of paragraph 20, wherein the dose is administered at least twice.

22. The method of paragraph 21, wherein the second dose is administered as a booster.

23. The method of paragraph 21, wherein each dose comprises an amount of the composition that does not induce immunity alone and wherein the cumulative effect of at least two doses of the composition results in immunity to Vibrio cholerae.

24. The method of paragraph 19, wherein the administering does not require sodium bicarbonate in potable water.

### EXAMPLES

### Example 1: A Self-Assembling Whole Cell Vaccine Antigen Presentation Platform

### Summary

Provided herein is a novel, customizable and self-assembling vaccine platform that exploits the Vibrio cholerae bacterial biofilm matrix for antigen presentation. This technology was used to create a proof-of-concept, live-attenuated whole cell vaccine that is boosted by spontaneous association of a secreted protein antigen with the cell surface. Sublingual administration of this live-attenuated vaccine to mice confers protection against V. cholerae challenge and elicits production of antigen-specific stool IgA. The platform presented herein enables development of antigen-boosted vaccines that are simple to produce and deliver, addressing many of the obstacles to vaccination against diarrheal diseases. This can also serve as a paradigm for the development of broadly protective, biofilm-based vaccines against other mucosal infections.

### Background

Intestinal pathogens are responsible for 1.7 billion cases of childhood disease annually with profound consequences for physical and cognitive development in resource-poor settings (1). While treatment of infection is essential for survival, prevention is preferable as it allows children to escape life-altering developmental sequelae. In the absence of improved sanitation, vaccination is an excellent means of prevention. However, few viable options exist for vaccination against diarrheal infections.

Vibrio cholerae, the bacterium responsible for the severe diarrheal disease cholera, forms multi-layer structures or biofilms on surfaces by secreting a Vibrio polysaccharide (VPS)-based matrix that remains tightly associated with the cell (2-4). When provided with the requisite nutritional signals in growth media, even free-swimming or planktonic V. cholerae synthesize a matrix with components similar to those in biofilms (4, 5). A proteomic approach was previously employed to identify proteins in the V. cholerae biofilm matrix (6). One of these proteins, RbmA, is a lectin that reinforces intercellular attachments by binding to VPS (6-8). It was previously proposed that RbmA could be used to non-covalently link secreted proteins to the cell surface (5). In this study, it is shown that fusion of a secreted protein, the B subunit of cholera toxin (CTB), to the Vibrio cholerae biofilm matrix protein RbmA leads to decoration of the surface of planktonic cells with this antigen. Sublingual administration of this antigen-decorated, whole cell vaccine to mice elicits an immune response both to the V. cholerae O antigen and CTB and passively protects infant mice against cholera challenge. While this study uses the biofilm matrix produced by V. cholerae for antigen presentation in the vaccine, it is proposed that this technology can be applied to any bacterium that forms a biofilm and can be used to induce protective immunity against diverse mucosal infections. Furthermore, a self-assembling, flexible protein antigen presentation platform was developed that can be used to create an affordable combination vaccine targeting diarrheal disease.

### Results

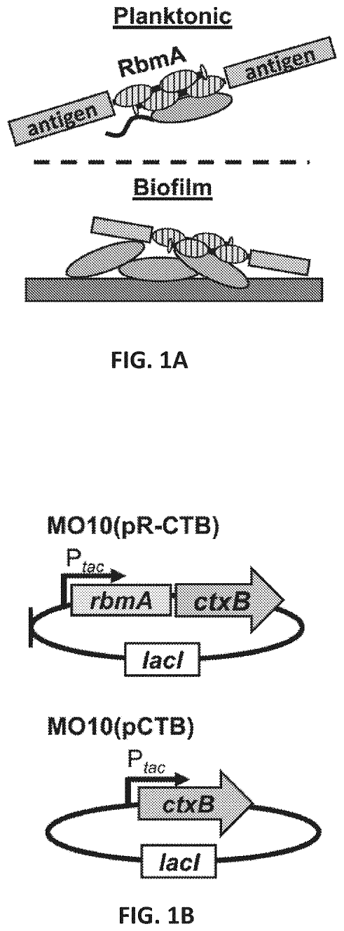

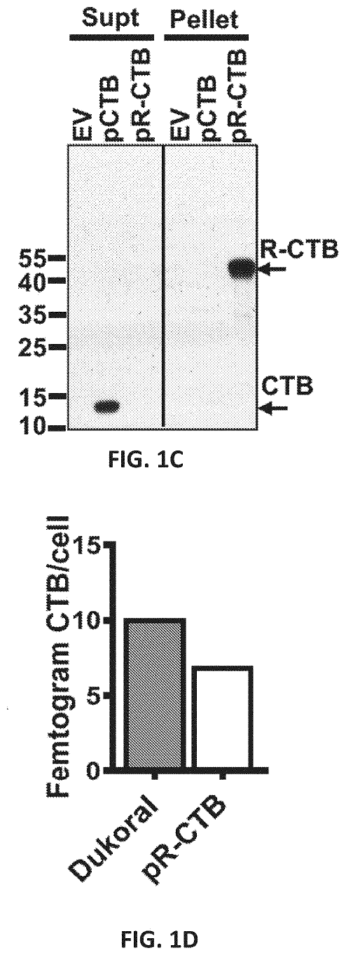

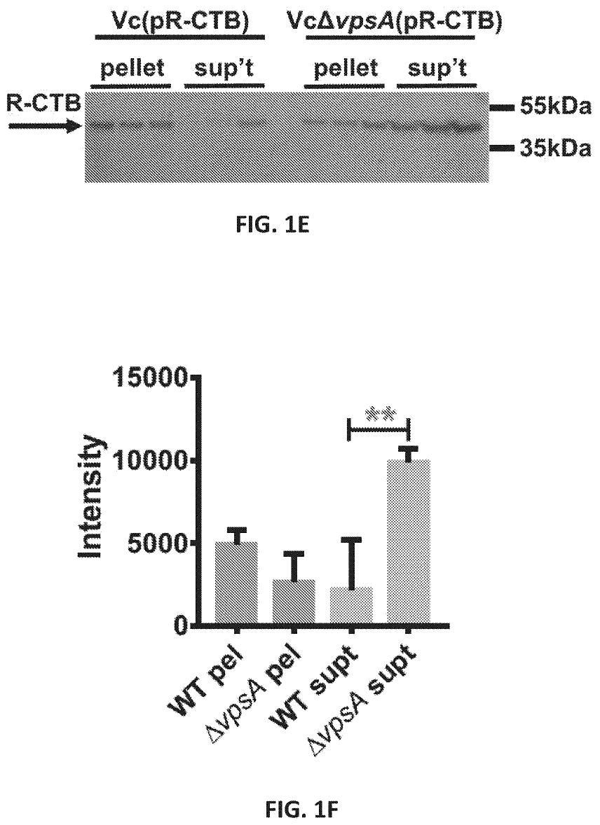

Fusion of the B subunit of cholera toxin (CTB) to the C-terminus of RbmA results in antigen secretion and association with the cell surface. RbmA, a lectin that spontaneously associates with the V. cholerae biofilm matrix polysaccharide (VPS) after secretion from the cell, consists of two tandem fibronectin III domains (9, 10). These domains bind to VPS as an antiparallel homodimer to mediate cell-to-cell adhesion (6, 8, 9). While RbmA is essential for biofilm structure and development, V. cholerae expresses VPS and RbmA in the free-swimming or planktonic state when cultured in LB broth (4, 5). It was previously demonstrated by the inventors that secreted proteins can be anchored to the surface of planktonic cells by genetic fusion to the C terminus of RbmA (FIG. 1A) (5). To demonstrate the utility of fusion to RbmA as means of antigen presentation, a prototype vaccine was generated that harbors a plasmid expressing CTB fused to the C terminus of RbmA (R-CTB) under the control of an inducible promoter (FIG. 1B, Table 1). A similar strain harboring a plasmid encoding native CTB was used as a control.

TABLE 1
Strains and plasmids used in this study.

| | Description | Reference |
|---|---|---|
| Strains/Plasmid | | |
| V. cholerae | | |
| PW17 | N16961 (El Tor, Inaba); Sm^{r} | (1) |
| PW724 | MO10; Sm^{r} | (2) |
| PW839 | MO10ΔctxA; Sm^{r} | (3) |
| PW1289 | MO10ΔvpsA, Sm^{r} | (4) |
| PW2132 | MO10 carrying pFLAG-CTC- | This study |
| | rbm-ctxB, Sm^{r}, Ap^{r} | |
| PW2133 | MO10ΔvpsA carrying pFLAG- | This study |
| | CTC-rbm-ctxB, Sm^{r}, Ap^{r} | |
| E. coli | | |
| SM10λpir | thi thr leu tonA lacY supE | (5) |
| | recA::RP4-2-Tc::Mu | |
| | (λpirR6K); Km^{r} | |
| Plasmids | | |
| pWM91 | oriR6K mobRP4 lacI pTac tnp | (6) |
| | mini-Tn10; Km^{r} Ap^{r} | |
| pFLAG-CTC-rbmA-ctxB | IPTG-inducible expression of | (7) |
| | RbmA-CTB fusion protein; Ap^{r} | |

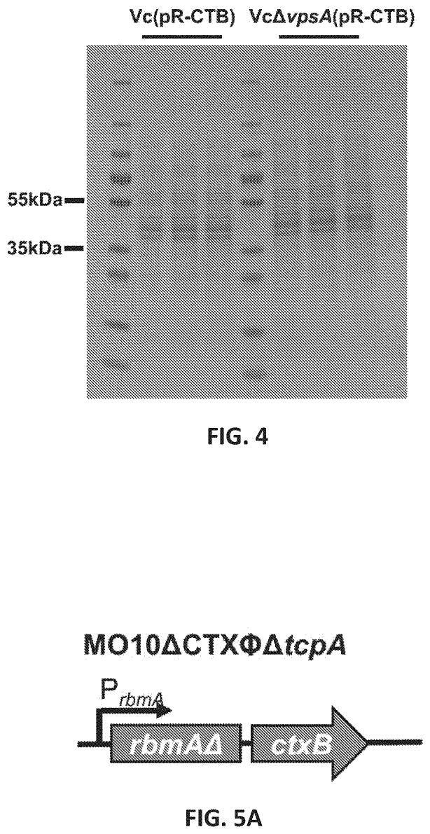

Quantitative Western blot analysis was used to measure association or R-CTB with cells. As shown in FIG. 1C, CTB was found in the cell pellet only in cells harboring the R-CTB fusion. The amount of R-CTB associated with cells compared favorably with an amount equivalent to that incorporated in Dukoral®, a licensed cholera vaccine comprised of inactivated whole V. cholerae and purified recombinant CTB (FIG. 1D). Previously, immunofluorescence was used to show that R-CTB is secreted and decorates the cell surface (5). To assess the proportion of synthesized R-CTB that is exported and becomes associated with VPS, R-CTB cell association in a parental V. cholerae strain was compared with that of a Δvps mutant. When equal amounts of total protein were loaded (FIG. 4), approximately four times more R-CTB was found in the supernatant in the absence of VPS, consistent with secretion from the cell (FIGS. 1E and 1F). However, a small fraction of R-CTB did remain cell associated, indicating retention in the cytoplasm or periplasm.

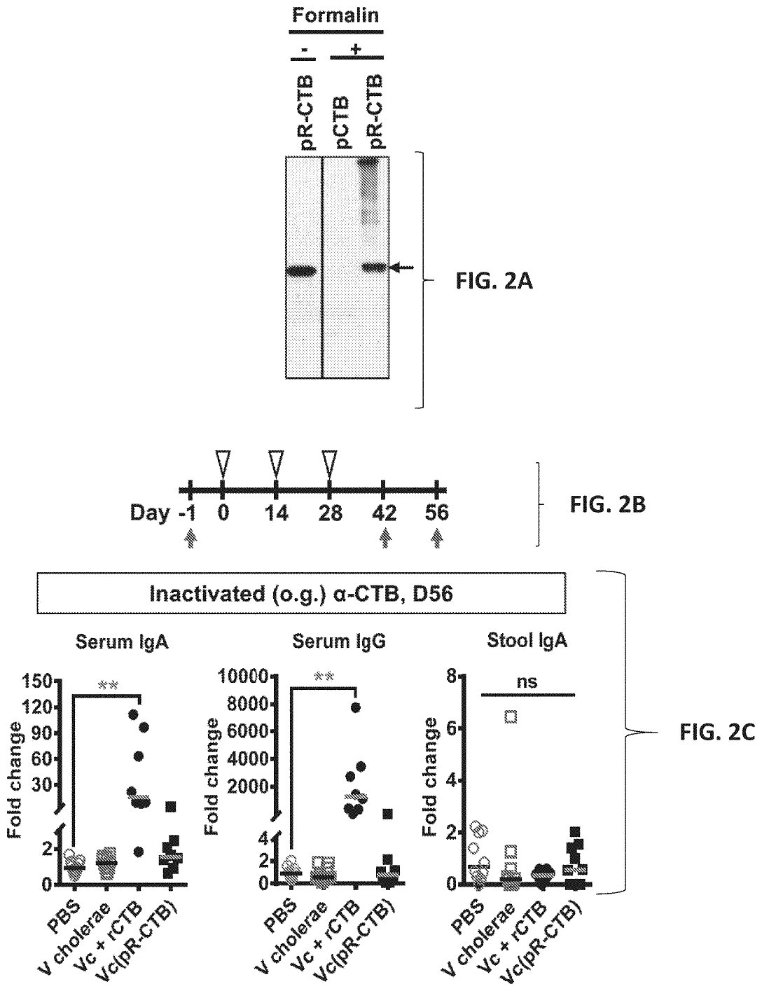

Dukoral®, a currently licensed cholera vaccine, consists of killed, whole V. cholerae cells combined with the purified B subunit of cholera toxin (CTB). This vaccine is costly and must be administered with sodium bicarbonate in a large volume of potable water to ensure presentation of intact CTB to the intestinal immune system. Due to its higher cost and requirement for potable water, Dukoral® is mainly a vaccine for travelers from affluent countries. To determine whether it was possible to create a vaccine presenting a comparable amount of CTB without the need for purification or administration with bicarbonate, a formalin-inactivated whole cell vaccine that expresses R-CTB was produced. While formalin treatment led to some protein cross-linking, CTB remained cell-associated (FIG. 2A). The fixed vaccine preparation was administered to mice by orogastric gavage (o.g.) with boosters 2 and 4 weeks later (FIG. 2B) and the immune response was assessed. As a positive control, a Dukoral-like vaccine consisting of formalin-fixed wild-type V. cholerae combined with purified CTB was prepared. Although not required for the fixed vaccine, both the R-CTB vaccine and the positive control were administered with bicarbonate to allow a direct comparison. Negative controls included PBS alone and formalin-fixed wild-type V. cholerae. While the Dukoral®-like vaccine induced production of CTB-specific serum IgA and IgG, the formalin-treated Vc(pR-CTB) vaccine elicited no antigen-specific antibodies (FIG. 2C). It was hypothesized that this was the result of formalin fixation of R-CTB, which has been reported to alter protein structure and abrogate antigenicity (11). Therefore, formalin fixation was eliminated and a live-attenuated formulation of the vaccine was produced.

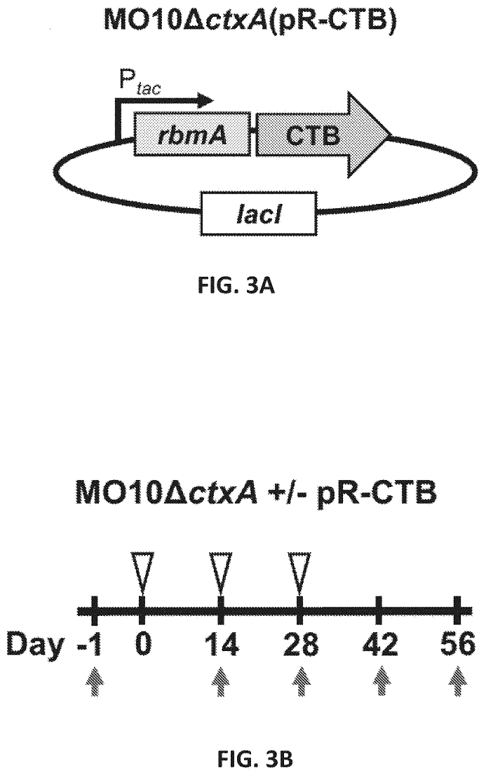

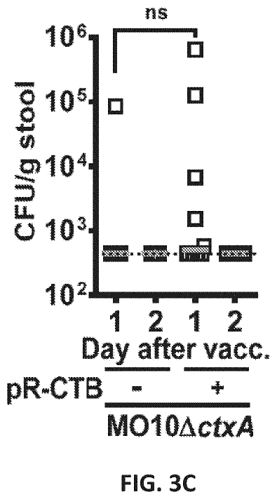

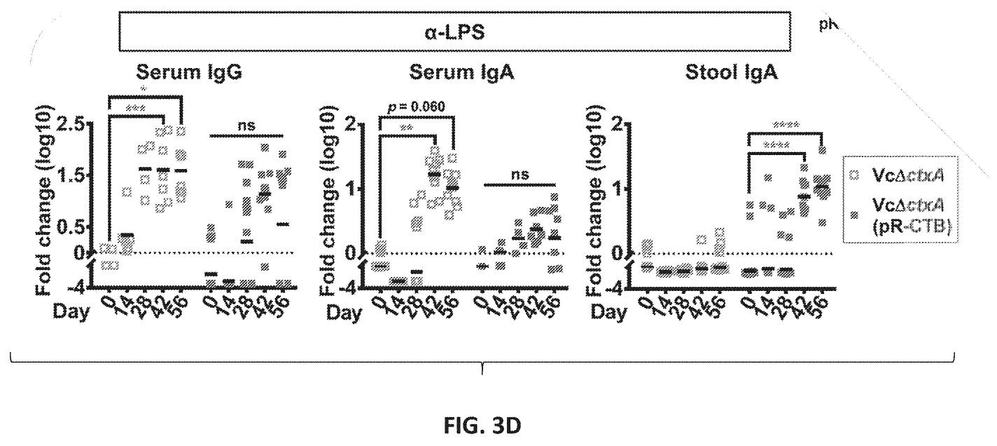

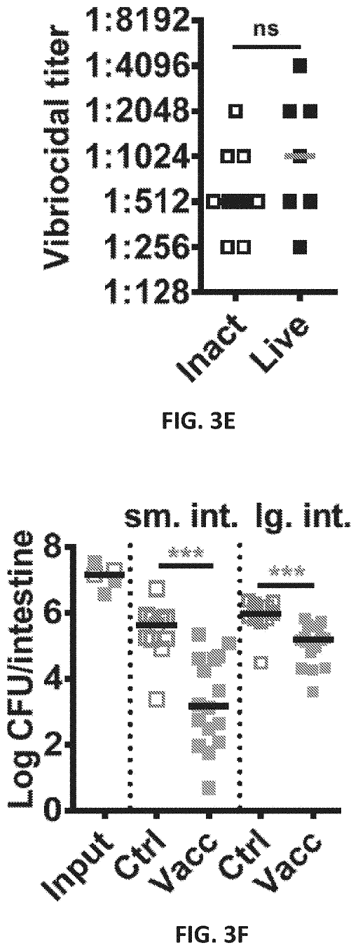

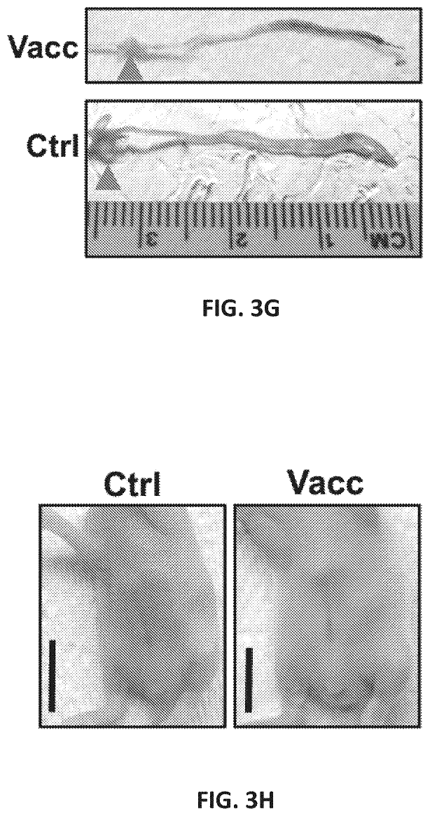

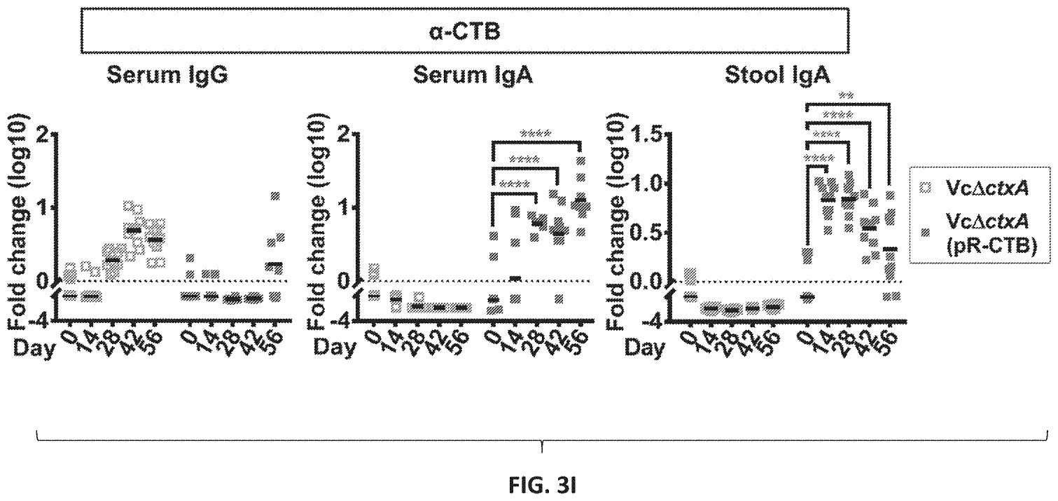

There are several live-attenuated cholera vaccines in various stages of development that are designed to be delivered orally (12-14). While the oral delivery route is effective for induction of an immune response at the intestinal mucosa, it also presents challenges. To maintain V. cholerae viability during transit through the stomach and, thus, ensure presentation of intact protein antigens to the intestinal innate immune system, oral vaccines must be administered with bicarbonate buffer. This demands a willing recipient and a reliable source of potable water, which may be difficult to obtain in resource-poor regions where these vaccines are most necessary. Furthermore, the possibility of intestinal colonization by live V. cholerae bacteria increases the risk of dissemination into the environment and may disrupt the normal intestinal microbiota. To circumvent these shortcomings, a live V. cholerae vaccine expressing R-CTB was administered via the sublingual route in a small volume of phosphate buffered saline (FIG. 3A) according to the schedule shown in FIG. 3B. This vaccine was attenuated by deletion of ctxA. Vaccine shedding was observed in a minority of animals and cleared rapidly, indicating little or no intestinal colonization (FIG. 3C). Sublingual administration of the R-CTB-expressing and control vaccines yielded a robust serum IgG response to V. cholerae LPS (FIG. 3D).

Furthermore, the day 42 serum vibriocidal titers, a measure of antibody-mediated activation of serum complement and a predictive marker for protection against disease, did not differ between the two vaccine groups (FIG. 3E). Inclusion of R-CTB increased LPS-specific serum and stool IgA, indicating enhancement of the mucosal immune response (FIG. 3D). CTB is known to upregulate TGF-β1, and this, in turn, has been shown to increase IgA isotype switching (15). Binding of CTB pentamers to gangliosides on the cell surface is believed to be essential for the immunomodulatory function of CTB. It seems unlikely that R-CTB, which is bound to the VPS, could form ganglioside-binding pentamers. Without wishing to be bound by theory, it is possible either that immunomodulation by CTB does not depend on pentamer formation or that cell-associated R-CTB potentiates the mucosal immune system by a mechanism distinct from that of CTB.

Because the immune response to LPS is correlated with protection against infection, the passive immunity afforded by the live-attenuated, sublingual vaccine was assessed using an infant mouse model of cholera. Suckling mice born to immunized or unimmunized dams were challenged with wild-type V. cholerae O139 and assessed for disease outcome after 24 hours (16, 17). Pups born to vaccinated dams had significantly decreased intestinal V. cholerae colonization (FIG. 3F), while animals in the unvaccinated group exhibited signs of cholera including fluid accumulation in the cecum and large intestine as well as reduced skin turgor indicative of dehydration (FIGS. 3G and 3H). Without wishing to be bound by theory, the inventors conclude that sublingual administration of a live, attenuated vaccine to dams induces an adaptive immune response that affords passive protection to offspring against cholera.

Sublingual administration of a live, attenuated vaccine expressing R-CTB induces an IgA-specific immune response to CTB. The immune response to CTB presented on the cell surface by fusion to RbmA was measured. When compared to vaccination with live, attenuated V. cholerae ΔctxA alone, vaccination with ΔctxA(pR-CTB) yielded a rapid systemic and mucosal CTB-specific IgA response as detected in the serum and stool, respectively (FIG. 3I, Table 2), while serum IgG was not detected throughout the study period. This was consistent with a previous report that sublingual immunization with a live bacterial vector generated antigen-specific fecal IgA but not serum IgG (18).

TABLE 2
Statistical analysis of fold change in antigen-specific antibody production
between the control (VcΔctxA) and vaccine (VcΔctxA(pR-CTB))
groups (FIGs. 2D and 2H).

| | VcΔctxA vs. VcΔctxA(pR-CTB) | | | |
|---|---|---|---|---|
| | D14 | D28 | D42 | D56 |
| LPS-specific | | | | |
| antibody | | | | |
| Serum IgG | Not | Not | n.s. | n.s. |
| | determined | determined | | |
| Serum IgA | Not | Not | n.s. | n.s. |
| | determined | determined | | |
| Stool IgA | n.s. | n.s. | **** | **** |
| CTB-specific | | | | |
| antibody | | | | |
| Serum IgG | Not | Not | Not | Not |
| | determined | determined | determined | determined |
| Serum IgA | Not | **** | **** | **** |
| | determined | | | |
| Stool IgA | **** | **** | **** | **** |

Statistical significance between vaccine groups was not assessed (not determined) at time points where neither group exhibited significant fold change in antibody production compared with the pre-immune levels.
**** p ≤ 0.0001 using one-way ANOVA followed by Tukey's test.

These results demonstrate for the first time that a protein antigen anchored to the biofilm matrix of a bacterial cell can be delivered sublingually to stimulate an antigen-specific mucosal immune response.

Efficacious, simply prepared, and easily administered vaccines that provide immunological protection against multiple mucosal infections could greatly enhance childhood health worldwide. This study provides two significant advances towards this goal. First, the feasibility of fusing a secreted protein antigen to the V. cholerae biofilm matrix protein RbmA as a means of decorating the surfaces of whole V. cholerae cells was assessed. Second, it was demonstrated that sublingual delivery of such antigen-decorated cells elicits a systemic and mucosal immune response to this antigen. This study shows that sublingual immunization with a live-attenuated V. cholerae vaccine provides passive protection against cholera in a murine model.

Heterologous antigen presentation in whole cell V. cholerae vaccines has previously relied on plasmid-based protein expression (19-22). This requires live cells to reach the target site, often the intestinal mucosa, and express the antigen in situ. Described herein is a vaccine platform in which heterologous antigens can be accumulated on the cell surface prior to delivery. Therefore, while the described prototype vaccine was delivered in live attenuated form, it may also be amenable to delivery as a inactivated vaccine, which would increase the shelf life and safety. Although orogastric delivery of a formalin-inactivated vaccine did not produce CTB-specific antibodies, it is possible that a shorter exposure time to formalin or a different method of inactivation could minimize deleterious effects on antigenicity. Therefore, optimization of vaccine formulation and delivery is an imperative direction of future research.

While the vaccine was administered sublingually, it is possible that the immune response observed herein resulted from presentation of antigens to the intestinal or nasal mucosa. While previous investigators showed that protein-based vaccines delivered in a 15 μl volume or less remained localized to the sublingual space (23), vaccine shedding in the feces was observed 24 hours after delivery in a few animals. This indicates that the vaccine cells were swallowed either at the time of administration or at some time thereafter. Given the propensity of viable V. cholerae to persist on diverse surfaces, such an occurrence would be difficult to prevent. A more precise test of the efficacy of the sublingual route awaits the development of an inactivated formulation of this vaccine.

Provided herein is a vaccine platform based on the V. cholerae biofilm matrix that elicits an immune response against protein antigens of a diarrheal pathogen when applied to the sublingual space. Proteins and lectins, in particular, are a common if not universal component of bacterial biofilm matrices (6, 24-26). Furthermore, sublingual delivery of protein antigens has been shown to generate mucosal immune responses in the intestine, lungs, and female genital tract (23, 27, 28). Therefore, it is specifically contemplated herein that this technology can be generalizable to the biofilm matrices synthesized by pulmonary or sexually-transmitted bacterial pathogens, and that these biofilm matrix components may be similarly harnessed for presentation of heterologous antigens relevant to these mucosal surfaces.

### Materials and Methods

Bacterial Strains and Culture Conditions.

Vibrio cholerae strains were cultured in Luria-Bertani (LB) broth supplemented with 100 μg/mL streptomycin at 27° C., with shaking at 200 revolutions per minute. Escherichia coli was grown in LB broth at 37° C. with shaking. Where necessary, plasmids were maintained with 100 μg/mL of ampicillin in the culture medium. Protein production from plasmid-borne P_{tac} was induced with 0.5 mM IPTG (β-d-l-thiogalactopyranoside). Frozen stocks were maintained in 15% glycerol at −80° C. Strains used in this study are listed in Table 1.

Construction of Prototype Vaccines Expressing Plasmid-Encoded R-CTB.

The previously described pFLAG-CTC derivative carrying the gene encoding the B subunit of cholera toxin (CTB) fused to the gene encoding RbmA (R-CTB) (5) or CTB alone under an IPTG-inducible promoter was introduced into wild-type MO10, ΔctxA, or ΔvpsA by electroporation. Protein production in positive transformants was verified by Western blot analysis using an anti-CTB antibody as described below.

Electroporation.

Vibrio cholerae strains were inoculated into 30 mL of LB broth and incubated at 22° C. until an optical density of 0.3 was reached. Cultures were chilled on ice for 20 min and then collected by centrifugation at 8,000×g for 10 min at 4° C. Cells were washed twice in 13 mL of cold 2 mM CaCl₂, once in 2 mL of cold 10% glycerol, and then collected by centrifugation at 6,000×g for 10 min. The cells were finally resuspended in 10% glycerol and electroporated in 50 μL aliquots by applying 1.8 kV to a 0.1 cm gap cuvette. Cells were immediately recovered by adding 250 μL of LB medium and incubating for 1 hr at 37° C. with shaking at 200 rpm. Transformants were selected on LB agar containing 100 μg/mL of ampicillin and confirmed by PCR and Western blot.

### Vaccine Preparation

Protein Induction.

Frozen stocks were inoculated into 3 mL of LB supplemented with streptomycin and ampicillin (LB-Sm/Amp) and cultured at 27° C. overnight. This starter culture was collected by centrifugation, washed once with LB-Sm/Amp, and sub-cultured into 25 mL of fresh LB-Sm/Amp in a 250-mL flask. After incubating for 6-8 h at 27° C. with shaking, IPTG was added to a final concentration of 0.5 mM, and the culture was incubated for an additional 10 h at 27° C. with shaking.

Trichloroacetic Acid (TCA) Precipitation of Secreted Proteins.

Proteins secreted into the supernatant were precipitated with TCA and washed with acetone. Briefly, spent supernatant was passed through a 0.2 μm filter to remove bacterial cells. TCA was added to the cell-free supernatant to a final concentration of 10% and the supernatant was incubated overnight at 4° C. with gentle mixing.

Precipitated Proteins were Collected by Centrifugation and Washed Three Times with Ice-Cold Acetone.

Residual acetone was evaporated by brief incubation at 95° C. The protein pellet was resuspended in 4× Laemmli buffer containing β-mercaptoethanol and prepared for Western blot as described below.

Preparation of Whole Cell Vaccines.

After protein induction, the bacterial culture was centrifuged at 5,000×g for 15 min at 4° C. to collect cells. The supernatant was passed through a 0.2 μm filter, and the resulting cell-free supernatant was used for Western blot analysis. The remaining bacterial pellet was washed three times with 12 mL of sterile PBS and finally resuspended in 1 mL of PBS. This constituted the live, whole cell vaccine. For each vaccine preparation, 10 μL was removed to quantify colony forming units (CFU), and 20 μL was reserved for Western blot analysis. For each immunization, the vaccine was prepared and used within 2 h.

Western Blot Analysis.

Supernatants and cell pellet samples were separated by centrifugation. TCA precipitation of the supernatant was performed prior to detection of CTB. These samples were combined with 4× Laemmli buffer containing β-mercaptoethanol, sonicated in an ice bath, boiled for 5 min, and finally briefly centrifuged to remove particulates. Proteins were resolved on a denaturing 4-20% gradient Tris-HCl gel and then transferred onto a polyvinylidene difluoride membrane by semi-dry transfer (BioRad). The membrane was blocked in tris-buffered saline with 0.1% Tween (TBS-T) and 5% skim milk for 2 h at room temperature with gentle shaking. Fresh blocking solution containing primary antibody was added in a 1:1,000 dilution. An anti-CTB polyclonal antibody conjugated to horseradish peroxidase (HRP) (Pierce, PA1-85293) was used to detect RbmA-CTB in cell pellets and native CTB in supernatants. After overnight incubation with primary antibodies, the membrane was washed 3 times with TBS-T. All membranes were developed using an ECL Western blotting substrate (Pierce).

Quantification of R-CTB by Densitometry.

Known concentrations of purified CTB (List Laboratories) were resolved by SDS-PAGE alongside R-CTB samples and used as standards for quantification. ImageJ was used to generate a standard curve fitted to the intensities of bands corresponding to the CTB standards. The concentration of R-CTB was calculated using the linear portion of the standard curve.

Coomassie Staining.

To ensure equal loading for Western blots, a duplicate gel was run in parallel and stained with Imperial Protein Stain (Thermo Fisher) according to manufacturer's instructions and destained with nanopure water.

### Immunization and Sample Collection

Animals.

Female, 6-8-week old BALB/c mice were used in all immunization experiments. For sublingual immunizations, mice were purchased from Charles River Laboratories and housed in a biosafety level two facility at Boston Children's Hospital with food and water ad libitum. Mice were acclimatized for 5 days. All procedures had been previously approved by the Institutional Animal Care and Use Committee.

Sublingual Administration of Live, Attenuated Vaccine.

Mice were anesthetized by intraperitoneal injection with a cocktail of ketamine (100 mg/kg) and xylazine (10 mg/kg), then held upright while 10 μL of the vaccine was delivered under the tongue by a micropipette directed toward the floor of the mouth. Mice were maintained in the upright position for 2 min before resting, ventral side down, for at least 30 min until regaining consciousness.

Collection of Blood and Stool Samples.

Blood and stool samples were collected one day before vaccination and at the designated time points throughout the study period. Fresh stool pellets were frozen at −80° C. until use. Blood was collected from the tail vein using capillary tubes with clot activator (Sarstedt). Sera were obtained by clearing the clotted blood with centrifugation at 10,000×g for 5 min at room temperature and stored at −20° C. Stool samples were prepared as previously described (29). Briefly, pellets were thawed on ice, transferred to 15-mL conical tubes containing 3 mL of chilled resuspension solution (0.1 mg/mL soybean trypsin inhibitor, 3:1 mixture of PBS to 0.1 M EDTA), thoroughly homogenized, and centrifuged at 650×g for 10 min at room temperature. The supernatant was collected and centrifuged once more at 15,300×g for 10 min at 4° C. PMSF (phenylmethane sulfonyl fluoride) was added to the supernatant to a final concentration of 2 mM. Stool samples were kept at −20° C. or at −80° C. for long term storage.

Enumeration of V. cholerae in Fecal Pellets.

A fresh stool pellet was collected from each mouse 24 h and 48 h after sublingual immunization. The pellet was weighed, homogenized in 1 mL sterile PBS, and serially diluted. One hundred microliter of the undiluted and diluted stool suspensions were plated on LB agar containing 100 μg/mL streptomycin and incubated at 37° C. overnight. The number of colony forming units (CFU) was recorded and normalized to the weight of the pellet to calculate CFU per gram of stool. One tenth of the total stool suspension was plated, which contained approximately 22.7 mg stool/ml. The lower limit of detection was estimated to be approximately 440 CFU/g.

### Enzyme-Linked Immunosorbent Assays

### Quantification of CTB-Specific Antibodies by ELISA.

Standard Curve:

CTB-specific IgA was not available for use in a standard curve. Therefore, to assess linearity, standard curves were generated by capturing IgA and IgG in reference mouse serum with goat anti-mouse IgG or IgA. Microtiter plate wells were incubated overnight with 100 μg of goat anti-mouse IgG or IgA diluted in sodium carbonate buffer. The wells were washed in PBS-T and blocked with PBS-BSA. Reference mouse serum (Bethyl Laboratory) was diluted to 1 μg/mL of total IgG or IgA and applied to the wells. The wells were washed after overnight incubation, then probed with HRP-conjugated goat anti-mouse antibodies and developed as described above for quantification of CTB.

Test Samples:

Microtiter plates were coated with GM1 followed by purified CTB as described above. The plates were blocked in PBS-BSA and washed in PBS-T. Serially diluted sera or stool samples were applied to the wells and incubated overnight. Serum dilutions ranged from 1:50 to 1:6400, and stool dilutions ranged from 1:2 to 1:128. The plates were probed and developed as described above.

Quantification of Total Stool IgA by ELISA.

Total fecal IgA was used to normalize antigen-specific IgA in the stool. Each well of a microtiter plates was coated with 100 ng of goat anti-mouse IgA antibody in sodium bicarbonate buffer and incubated overnight. Plates were washed in PBS-T and blocked in PBS-BSA. Stool samples were serially diluted from 1:200 to 1:25,600 in PBS-T-BSA and added to the plates. The plates were incubated overnight and then probed and developed as described above.

Standard Curves were Generated as Described for CTB-Specific Antibodies.

Lipopolysaccharide (LPS) extraction and measurement of O-antigen specific antibodies. LPS was extracted from 50 mL of Vibrio cholerae MO10 (serotype O139) and N16961 (serotype O1) overnight cultures using a commercial kit (Bulldog Bio). Serum and stool antibodies recognizing the O1 or O139 serotypes were quantified as previously described (30). A 1:1,000 dilution of LPS in sodium carbonate buffer was applied to microtiter plates and incubated overnight. The plates were washed in PBS-T and blocked for 40 min at 37° C. in PBS-BSA. Serum and stool samples were applied to the plates in dilutions similar to those used to measure CTB-specific antibodies. The plates were incubated for 90 min at 37° C. and then washed in PBS-T. Plates were incubated for 90 min at 37° C. after addition of 100 ng of HRP-conjugated goat anti-mouse antibodies per well. Plates were developed using the same protocol described for quantification of CTB-specific antibodies.

Serum Vibriocidal Titers.

Serum vibriocidal antibody titers were determined as previously described with the following modifications (31). Immunized mouse sera were incubating at 56° C. for 1 h to inactivate endogenous complement, serially diluted two-fold in PBS in 0.5-μL tubes, and kept on ice. Wild-type MO10 was grown to mid-logarithmic phase in brain heart infusion broth containing 100 μg/mL streptomycin and diluted in PBS containing 20% guinea pig complement to 4×106 CFU/mL. An equal volume of this suspension was added to the serum dilutions to obtain a final concentration of 10% complement and 2×10⁶ CFU/mL V. cholerae, the mixture was incubated for 1 h at 37° C. with shaking at 200 rpm, and viable cells were enumerated by plating. Bactericidal titer was determined as the reciprocal of the serum dilution capable of killing 50% or more of the indicator strain compared with a control containing pre-immune or PBS-immunized serum. Sera from mice that received the inactivated vaccine were randomly pooled into groups of three for determination of vibriocidal titers.

Infant Mouse Challenge Model.

Orogastric challenge of infant mice. At the end of the study period (between 60 to 70 days after the initial immunization), vaccinated female mice were mated with age-matched male. Non-vaccinated, non-timed pregnant mice were purchased from Charles River Laboratories and housed in the same facility as vaccinated mice. When pups were born, wild-type MO10 was grown overnight in LB broth with 100 μg/mL streptomycin at 27° C. The cell density was adjusted to 5×10⁹ CFU/mL. Bacteria were collected by centrifugation at 6,000×g for 5 min and resuspended in 2.5% sodium bicarbonate (0.29 M). Four- to five-day-old pups were challenged with 2.5×10⁷ CFU of wild-type MO10 delivered in 50 μL of sodium bicarbonate solution by oral gavage. Pups were monitored for immediate signs of distress and then returned to the dam. All pups were sacrificed 24 h after infection, and signs of disease were documented.

Quantification of bacterial colonization. The small and large intestines were weighed, added to sterile conical tubes containing 1 mL of PBS, and homogenized. The homogenates were serially spread plated on LB agar supplemented with 100 μg/mL of streptomycin. Plates were incubated overnight at 37° C. The limit of detection for each spread plate is 10 CFU per intestine.

Statistical Analysis.

Statistical analyses were performed in GraphPad Prism 7. One-way ordinary ANOVA with Tukey's test was used for multiple comparisons. Two-tailed, unpaired Mann-Whitney test was used for pair-wise comparisons. All vaccine groups consisted of ten mice. Error bars indicate standard deviations unless otherwise noted. Western blot images are representative of experimental triplicates.

### REFERENCES

1. Kotloff K L, et al. 2013. Burden and aetiology of diarrhoeal disease in infants and young children in developing countries (the Global Enteric Multicenter Study, GEMS): a prospective, case-control study. The Lancet 382:209-222.
2. Watnick P I, Kolter R. 1999. Steps in the development of a Vibrio cholerae El Tor biofilm. Mol Microbiol 34:586-595.
3. Yildiz F H, Schoolnik G K. 1999. Vibrio cholerae O1 El Tor: identification of a gene cluster required for the rugose colony type, exopolysaccharide production, chlorine resistance, and biofilm formation. Proc Natl Acad Sci USA 96:4028-4033.
4. Smith D R, Maestre-Reyna M, Lee G, Gerard H, Wang A H J, Watnick P I. 2015. In situ proteolysis of the Vibrio cholerae matrix protein RbmA promotes biofilm recruitment. Proceedings of the National Academy of Sciences 112:10491-10496.
5. Absalon C, Ymele-Leki P, Watnick P I. 2012. The Bacterial Biofilm Matrix as a Platform for Protein Delivery. mBio 3:e00127-00112-e00127-00112.
6. Absalon C, van Dellen K, Watnick P I. 2011. A Communal Bacterial Adhesin Anchors Biofilm and Bystander Cells to Surfaces. PLoS Pathogens 7:e1002210.
7. Berk V, Fong J C, Dempsey G T, Develioglu O N, Zhuang X, Liphardt J, Yildiz F H, Chu S. 2012. Molecular architecture and assembly principles of Vibrio cholerae biofilms. Science 337:236-239.
8. Fong J C N, Karplus K, Schoolnik G K, Yildiz F H. 2006. Identification and characterization of RbmA, a Novel Protein Required for the Development of Rugose Colony Morphology and Biofilm Structure in Vibrio cholerae. Journal of Bacteriology 188:1049-1059.
9. Giglio K M, Fong J C, Yildiz F H, Sondermann H. 2013. Structural Basis for Biofilm Formation via the Vibrio cholerae Matrix Protein RbmA. Journal of Bacteriology 195:3277-3286.
10. Maestre-Reyna M, Wu W J, Wang A H. 2013. Structural insights into RbmA, a biofilm scaffolding protein of V. cholerae. PLoS One 8:e82458.
11. Aguilar P V, Fan Y-C, Chiu H-C, Chen L-K, Chang G-J J, Chiou S-S. 2015. Formalin Inactivation of Japanese Encephalitis Virus Vaccine Alters the Antigenicity and Immunogenicity of a Neutralization Epitope in Envelope Protein Domain III. PLOS Neglected Tropical Diseases 9:e0004167.
12. Perez J L, Garcia L, Talavera A, Oliva R, Valmaseda T, Ano G, Perez O, Sierra G. 2000. Passive protection of serum from volunteers inoculated with attenuated strain 638 of Vibrio cholerae O1 in animal models. Vaccine 19:376-384.
13. Talavera A, Ano G, Garcia H, Moreira T, Delgado H, Riveron L, Gil S, Miranda A, Cedre B, Valmaseda T, Pino Y, Perez J L, Infante J F, Garcia L, Sierra G. 2006. Process development for a Cuban cholera vaccine based on the attenuated strain Vibrio cholerae 638. Vaccine 24:3746-3749.
14. Chen W H, Cohen M B, Kirkpatrick B D, Brady R C, Galloway D, Gurwith M, Hall R H, Kessler R A, Lock M, Haney D, Lyon C E, Pasetti M F, Simon J K, Szabo F, Tennant S, Levine M M. 2016. Single-dose Live Oral Cholera Vaccine CVD 103-HgR Protects Against Human Experimental Infection With Vibrio cholerae O1 El Tor. Clin Infect Dis 62:1329-1335.
15. Royal J M, Matoba N. 2017. Therapeutic Potential of Cholera Toxin B Subunit for the Treatment of Inflammatory Diseases of the Mucosa. Toxins (Basel) 9.
16. Guentzel M N, Berry L J. 1974. Protection of Suckling Mice from Experimental Cholera by Maternal Immunization: Comparison of the Efficacy of Whole-Cell, Ribosomal-Derived, and Enterotoxin Immunogens. Infection and Immunity 10:167-172.
17. Sengupta D K, Boesman-Finkelstein M, Finkelstein R A. 1996. Antibody against the Capsule of Vibrio cholerae O139 Protects against Experimental Challenge. Infection and Immunity 64:343-345.
18. Ferreira P C D, da Silva J B, Piazza R M F, Eckmann L, Ho P L, Oliveira M L S. 2011. Immunization of Mice with Lactobacillus casei Expressing a Beta-Intimin Fragment Reduces Intestinal Colonization by Citrobacter rodentium. Clinical and Vaccine Immunology 18:1823-1833.
19. Butterton J R, Beattie D T, Gardel C L, Carroll P A, Hyman T, Killeen K P, Mekalanos J J, Calderwood S B. 1995. Heterologous Antigen Expression in Vibrio cholerae Vector Strains. Infection and Immunity 63:2689-2696.
20. Ryan E T, Crean T I, Kochi S K, John M, Luciano A A, Killeen K P, Klose K E, Calderwood S B. 2000. Development of a DglnA Balanced Lethal Plasmid System for Expression of Heterologous Antigens by Attenuated Vaccine Vector Strains of Vibrio cholerae. Infection and Immunity 68:221-226.
21. Silva A J, Eko F O, Benitez J A. 2008. Exploiting cholera vaccines as a versatile antigen delivery platform. Biotechnol Lett 30:571-579.
22. Vishwakarma V, Sahoo S S, Das S, Ray S, Hardt W D, Suar M. 2015. Cholera toxin-B (ctxB) antigen expressing Salmonella Typhimurium polyvalent vaccine exerts protective immune response against Vibrio cholerae infection. Vaccine 33:1880-1889.
23. Çuburu N, Kweon M-N, Song J-H, Hervouet C, Luci C, Sun J-B, Hofman P, Holmgren J, Anjuère F, Czerkinsky C. 2007. Sublingual immunization induces broad-based systemic and mucosal immune responses in mice. Vaccine 25:8598-8610.
24. Diggle S P, Stacey R E, Dodd C, Camara M, Williams P, Winzer K. 2006. The galactophilic lectin, LecA, contributes to biofilm development in Pseudomonas aeruginosa. Environ Microbiol 8:1095-1104.
25. Buttner H, Mack D, Rohde H. 2015. Structural basis of Staphylococcus epidermidis biofilm formation: mechanisms and molecular interactions. Front Cell Infect Microbiol 5:14.
26. Corrigan R M, Rigby D, Handley P, Foster T J. 2007. The role of Staphylococcus aureus surface protein SasG in adherence and biofilm formation. Microbiology 153:2435-2446.
27. Luo Q, Vickers T J, Fleckenstein J M, Staats H F. 2016. Immunogenicity and Protective Efficacy against Enterotoxigenic Escherichia coli Colonization following Intradermal, Sublingual, or Oral Vaccination with EtpA Adhesin. Clinical and Vaccine Immunology 23:628-637.
28. Cuburu N, Kweon M N, Hervouet C, Cha H R, Pang Y Y, Holmgren J, Stadler K, Schiller J T, Anjuere F, Czerkinsky C. 2009. Sublingual immunization with nonreplicating antigens induces antibody-forming cells and cytotoxic T cells in the female genital tract mucosa and protects against genital papillomavirus infection. J Immunol 183:7851-7859.
29. Ryan E T, Butterton J R, Zhang T, Baker M A, Stanley S L, Jr., Calderwood S B. 1997. Oral immunization with attenuated vaccine strains of Vibrio cholerae expressing a dodecapeptide repeat of the serine-rich Entamoeba histolytica protein fused to the cholera toxin B subunit induces systemic and mucosal antiamebic and anti-V. cholerae antibody responses in mice. Infect Immun 65:3118-3125.
30. Rollenhagen J E, Kalsy A, Saksena R, Sheikh A, Alam M M, Qadri F, Calderwood S B, Kovac P, Ryan E T. 2009. Transcutaneous immunization with a synthetic hexasaccharide-protein conjugate induces anti-Vibrio cholerae lipopolysaccharide responses in mice. Vaccine 27:4917-4922.
31. Qadri F, Mohi G, Hossain J, Azim T, Khan A M, Salam M A, Sack R B, Albert M J, Svennerholm A M. 1995. Comparison of the vibriocidal antibody response in cholera due to Vibrio cholerae O139 Bengal with the response in cholera due to Vibrio cholerae O1. Clin Diagn Lab Immunol 2:685-688.

### Example 2: Sublingual Antigen and Adjuvant Delivery in a Live-Attenuated Vaccine Platform by In Vivo mmCT Secretion

### Summary

The previous Example showed that, when a fusion protein comprised of the Vibrio cholerae biofilm matrix protein RbmA and the B subunit of cholera toxin (R-CTB) is expressed from a plasmid within V. cholerae, R-CTB is sequestered in the biofilm matrix. Sublingual delivery of these R-CTB-decorated cells as a vaccine results in a mucosal immune response to both V. cholerae LPS and CTB. It was proposed that antigen presentation on the cell surface through fusion to RbmA can serve as a platform for delivery of heterologous antigens. An adjuvant could greatly improve the magnitude and duration of the immune response to such a vaccine.

This study compares the immunogenicity of sublingually-delivered, live-attenuated V. cholerae vaccines expressing a chromosomally-encoded, mutant form of R-CTB containing R→A point mutations at positions 116 and 234 (cRL1-CTB) with or without a constitutively transcribed operon encoding the adjuvant mmCT, a previously described non-toxigenic form of cholera toxin. Sublingual administration of a vaccine encoding cRL1-CTB at the native RbmA locus elicits poor mucosal immune responses to both LPS and CTB, while a vaccine expressing the adjuvant mmCT alone elicits a rapid, robust, and long-lived response to both LPS and CTB. While joint delivery of mmCT and R-CTB blunts the immune response to CTB in a dose-dependent manner, mmCT is an excellent adjuvant for LPS. It is proposed herein that mmCT can also serve as an adjuvant for heterologous antigens delivered via this live-attenuated Vibrio cholerae vaccine platform.

### Background

V. cholerae is responsible for cholera, an epidemic diarrheal disease that targets children living in temperate climates and populations displaced by political unrest or environmental disaster such as those in Yemen, Rwanda, and Haiti (1-3). The two major virulence factors of V. cholerae are the toxin co-regulated pilus (TCP), which is required for colonization of the small intestine, and cholera toxin (CT), which produces the watery diarrhea characteristic of cholera (4). Both live-attenuated and inactivated whole cell vaccines that protect against cholera have been developed and are licensed (5-7). While pre-emptive and reactive vaccination has been shown to limit epidemic spread in such populations (8), practical considerations have limited the use of these vaccines (9, 10).

The existing live-attenuated and inactivated whole cell cholera vaccines are administered orogastrically in order to induce intestinal immunity. In comparison with parenteral immunizations, such mucosal immunizations more closely mimic natural infection and are amenable to large vaccination campaigns. However, because mucosal surfaces are constantly exposed to food and microbiota-derived antigens, they are by necessity tolerogenic environments. Mucosal adjuvants greatly improve the efficacy of these vaccines by promoting antigen uptake and enhancing the mucosal immune response (11-13). Cholera toxin (CT) is an excellent mucosal adjuvant, although its acute toxicity precludes clinical use. A detoxified form of cholera toxin, mmCT, which retains adjuvanticity but does not induce diarrhea, has recently been described (14, 15). Both CT and mmCT have been shown to enhance vaccine efficacy by promoting differentiation and maturation of Th17 cells (16-18), which closely correlates with production of mucosal IgA (19). However, these protein adjuvants must be purified, which complicates manufacturing, and given in a form that survives the acidity of the stomach, which complicates vaccine administration.

Example 1 describes a V. cholerae whole cell antigen presentation platform based on fusion of protein antigens to the biofilm matrix-associated protein RbmA. It was previously demonstrated that sublingual delivery of such a vaccine that expresses CTB fused to RbmA (R-CTB) from a plasmid induces a mucosal immune response to LPS and CTB and provides passive protection against cholera challenge in an infant mouse model (20). Vaccines that express a mutant form of R-CTB from the native RbmA promoter on the chromosome (cRL1-CTB), the adjuvant mmCT, or both were generated. The immune response to LPS and CTB elicited by sublingual delivery of these vaccines was compared. While a vaccine that expresses cRL1-CTB is poorly immunogenic, mmCT stimulates an excellent immune response to both V. cholerae LPS and CTB. Co-expression of mmCT and an RbmA-CTB fusion promotes dose-dependent tolerance to CTB but not LPS. Without wishing to be bound by theory, plasmid-based delivery of R-CTB appears to be the optimal choice for delivery of this antigen by an inactivated, whole cell vaccine that is unable to synthesize new proteins at the time of administration.

However, in vivo delivery of mmCT by a live-attenuated vaccine elicits a robust mucosal immune response to CTB and boosts the mucosal immune response to LPS. Therefore, when delivered in vivo by a live-attenuated V. cholerae vaccine platform, it is hypothesized that mmCT may serve as an adjuvant for unrelated antigens.

### Results

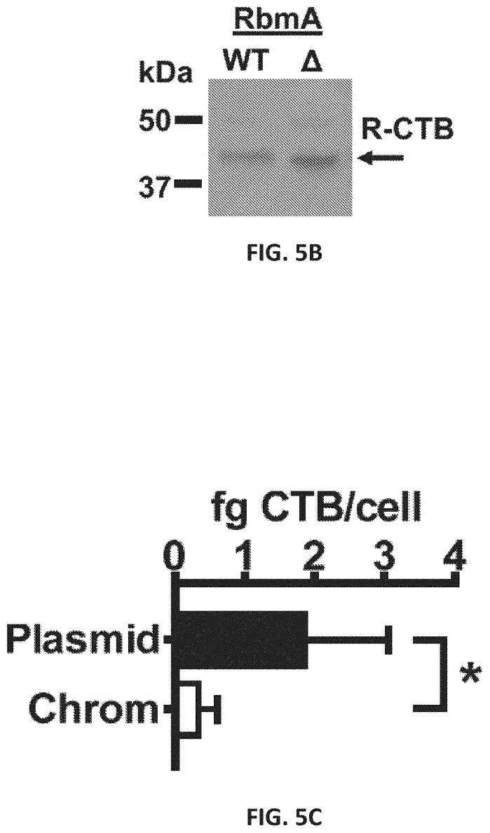

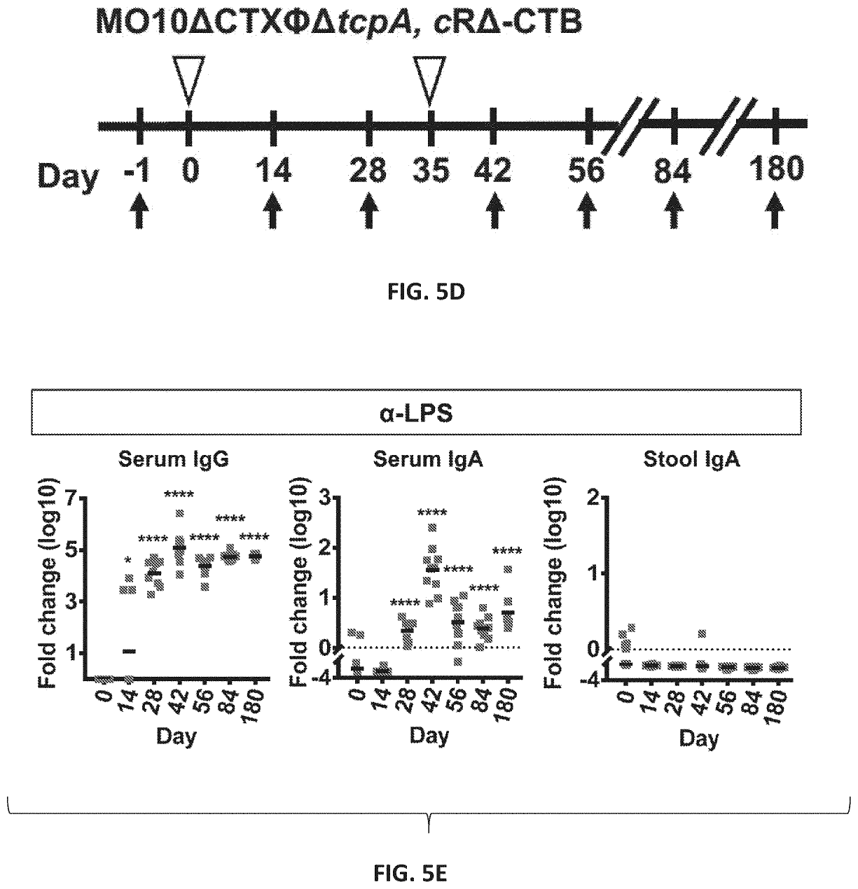

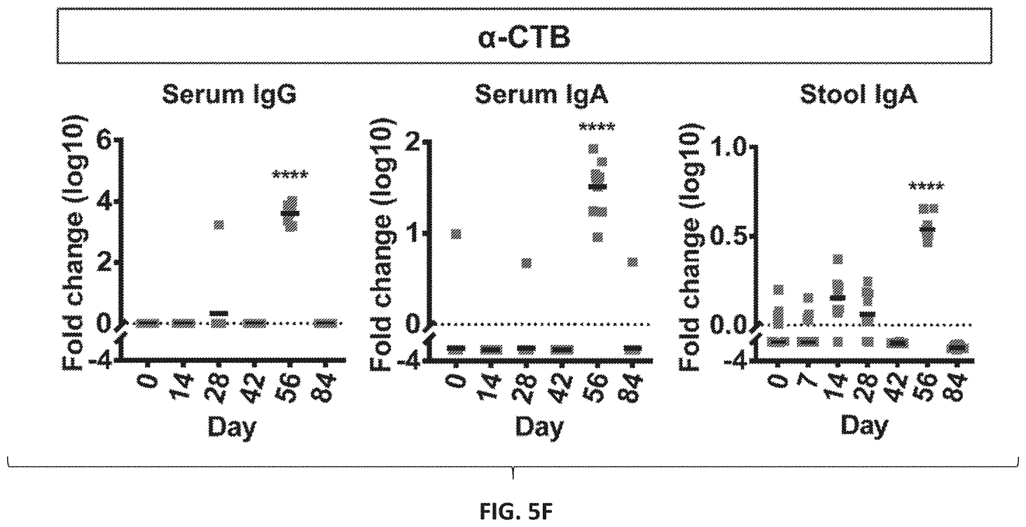

A live-attenuated vaccine expressing cRΔ-CTB results in poor mucosal responses to both V. cholerae LPS and CTB. To adapt the vaccine platform for safe delivery as a live-attenuated vaccine, the gene encoding R-CTB was moved from a plasmid to the native RbmA site of a V. cholerae strain lacking both the entire CTX phage and tcpA, the major component of TCP (FIG. 5A, Tables 3-5) (21). Additionally, two R→A mutations were introduced in RbmA at positions 116 and 234 (RΔ-CTB). These mutations have previously been found to abrogate the rugose phenotype in colonies (22). It was hypothesized that these mutations might reduce cell-cell interactions mediated by RbmA and, thus, alter antigen delivery to immune cells. Indeed, it was found that while RΔ-CTB remained associated with the cell pellets (FIG. 5B), biofilms formed under static conditions overnight by a strain carrying RΔ-CTB were more easily dispersed by agitation as compared with a strain carrying wild-type R-CTB (FIG. 10). This strain accumulated approximately 6-fold less RΔ-CTB on its surface as compared with a strain that expresses CTB conjugated to wild-type RbmA encoded on a plasmid (FIG. 5C). This strain was administered sublingually to mice with one booster. Blood and stool were collected at the time points shown in FIG. 5D. While LPS-specific serum IgG and IgA were detected, low levels of LPS-specific IgA in the stool indicated that the mucosal immune response to LPS was poor (FIG. 5E). In addition, the systemic and mucosal immune responses to CTB were measurable only after boosting (FIG. 5F). The vaccine strain carrying chromosomally encoded RΔ-CTB was poorly immunogenic as compared with previous observations of R-CTB expressed from a plasmid. This is attributed to decreased accumulation of RΔ-CTB on the cell surface, although one cannot rule out an effect of rbmA mutation.

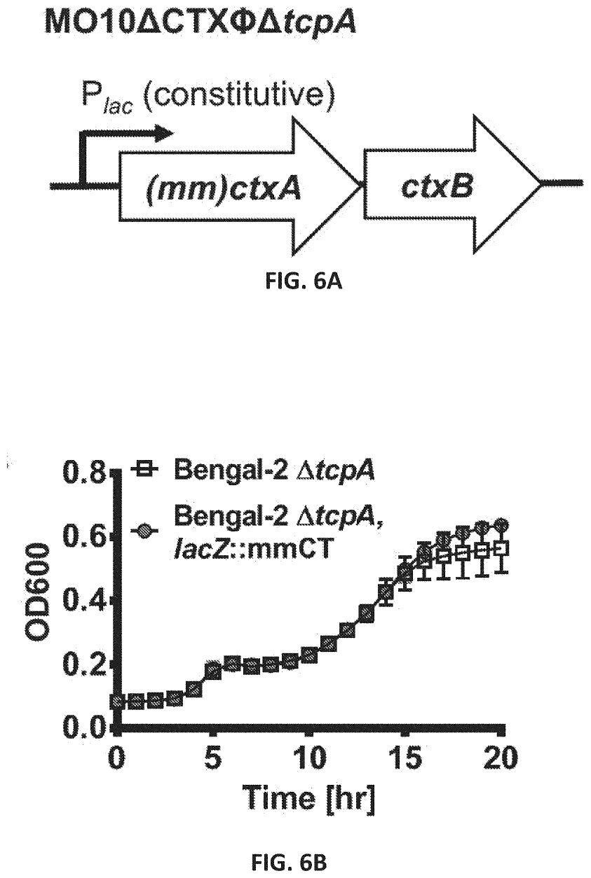

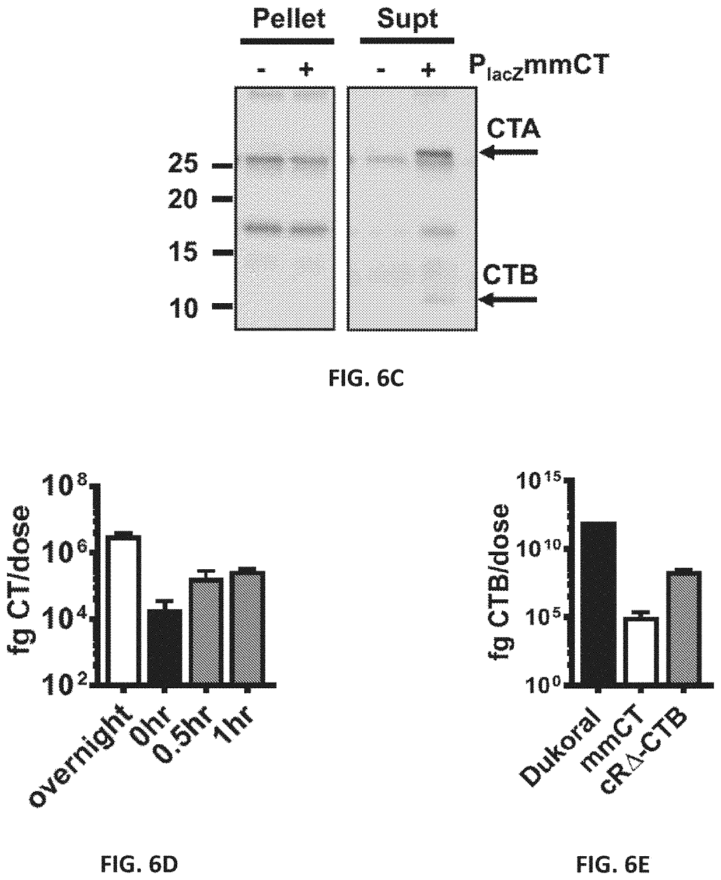

Immunization with an mmCT-producing vaccine results in a robust antibody response to CTB and augments the LPS-specific IgA response. Cholera toxin (CT) is an excellent but impractical mucosal adjuvant due to its toxicity. Recently, a multiply-mutated form of CT (mmCT) has been developed that retains adjuvanticity but has greatly reduced toxicity (15). It was hypothesized that mmCT might serve as an excellent adjuvant for the vaccines described herein. mmCT was introduced into the native lacZ site on the chromosome of V. cholerae, where expression is driven by the constitutive lacZ promoter (PlacZ::mmCT) (FIG. 6A). mmCT would be suboptimal as an adjuvant if in vivo expression reduced cell fitness. However, it was found that constitutive production of mmCT did not impair V. cholerae growth (FIG. 6B). Secretion of mmCT into the supernatant of bacteria cultured in LB broth was confirmed by Western blot using rabbit sera raised against cholera toxin (FIG. 6C). Using ELISA, it was determined that a 10 μL volume of the cell-free supernatant from an overnight culture of the mmCT-producing strain contained approximately 50 femtomoles of mmCT. Furthermore, after incubation in PBS at room temperature for 30 min and 1 hr, the length of time that the vaccine is estimated to persist in the murine sublingual space, approximately 3 femtomoles and 4.7 femtomoles of mmCT was detected per 10 μL volume, respectively (FIG. 6D). Even after the 1 hr incubation at room temperature in PBS, mmCT expression delivered approximately 4 orders of magnitude less CTB than is delivered by chromosomal expression of RL1-CTB and 8 orders of magnitude less CTB than the Dukoral® vaccine (FIG. 6E).

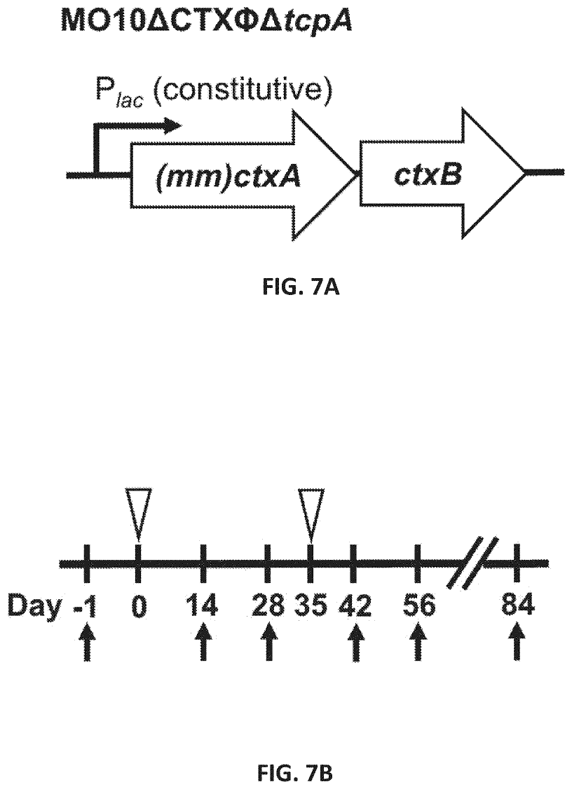

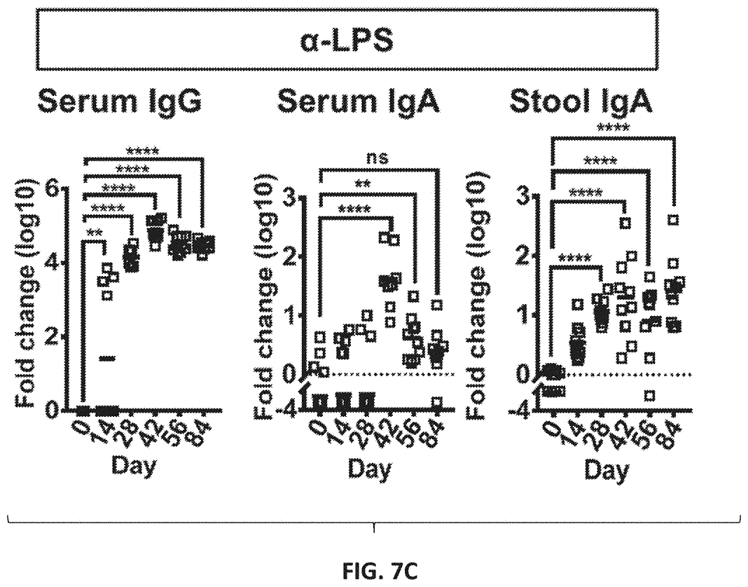

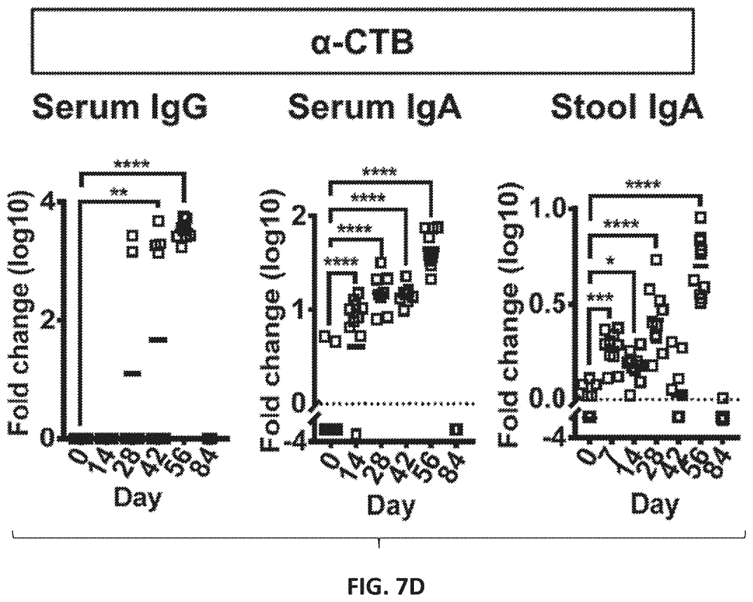

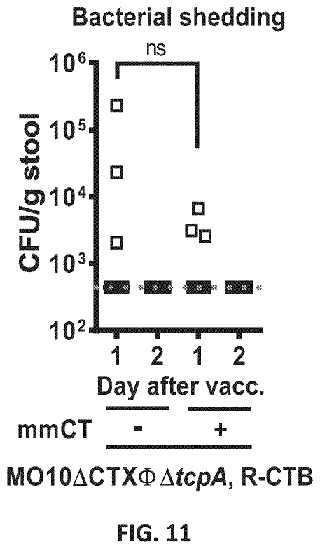

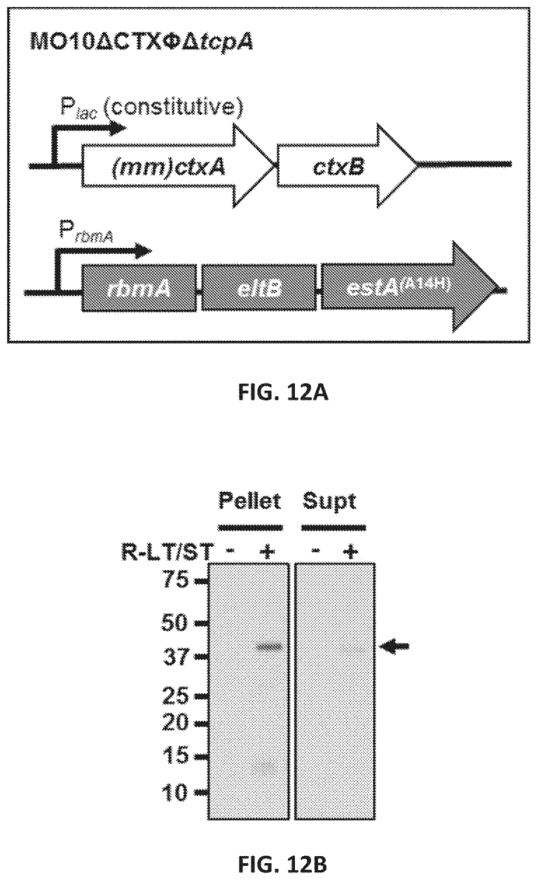

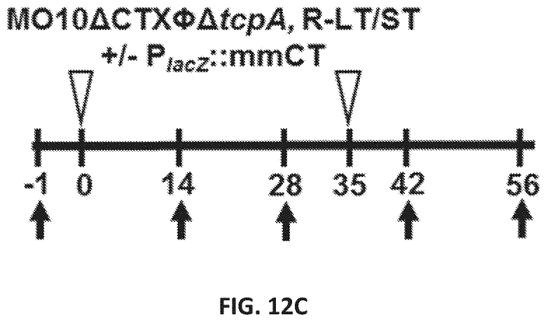

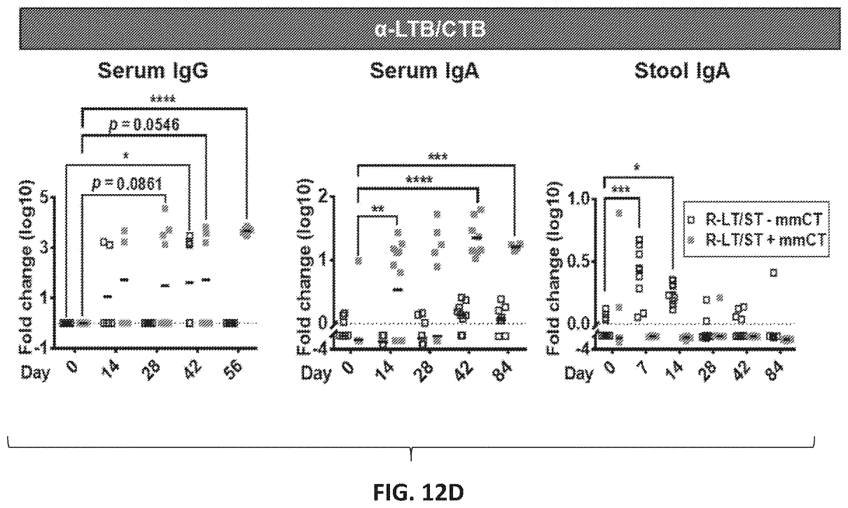

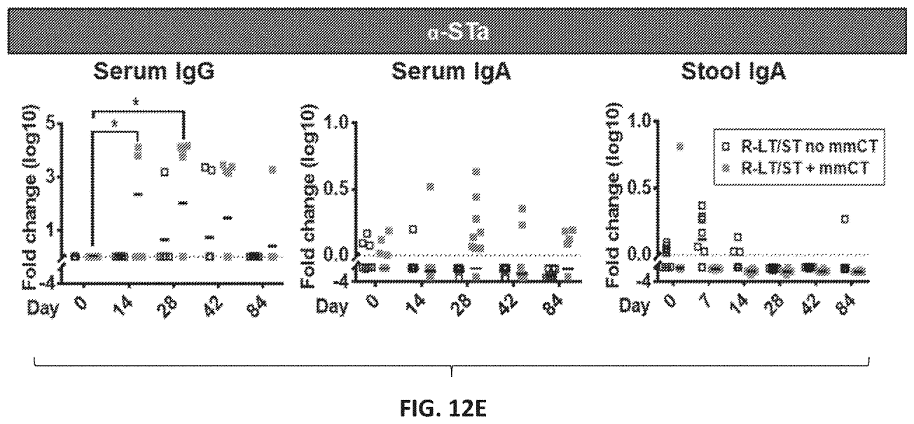

The live-attenuated vaccine expressing mmCT alone was administered to mice via the sublingual route (FIGS. 7A and 7B). Sublingual immunization with the live-attenuated vaccine greatly enhanced the mucosal immune response to LPS, which had not yet declined at day 84 after the primary immunization (FIGS. 7C and 11A). Serum and mucosal immune responses to CTB were robust and also lasted for several weeks but declined by day 84 (FIGS. 7D and 11B).

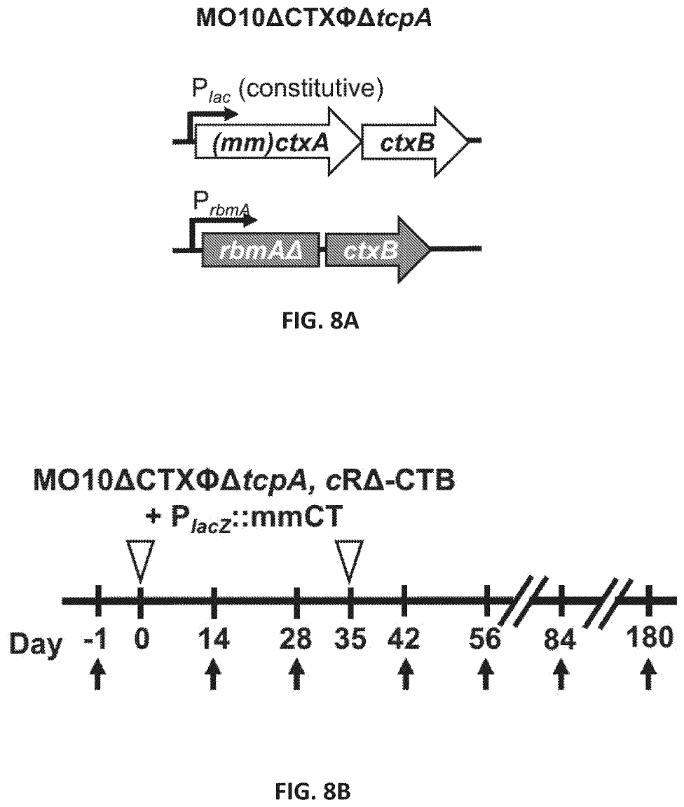

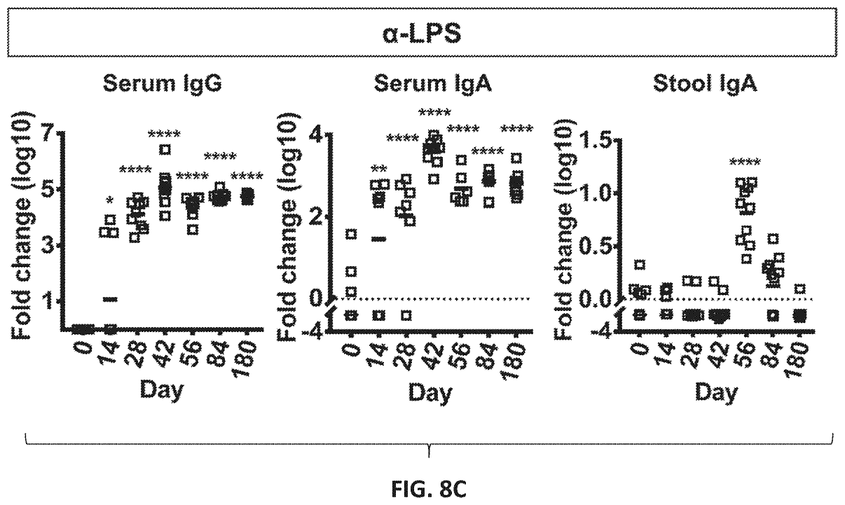

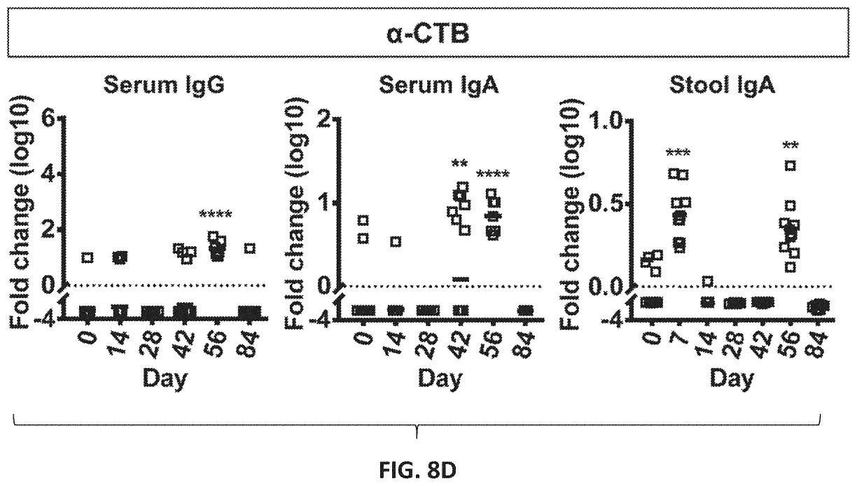

Co-delivery of mmCT and cRL1-CTB attenuates the immune response to CTB. Given the adjuvant activity of mmCT, it was questioned whether co-delivery of chromosomally-encoded cRL1-CTB and mmCT might further improve the immune response to CTB. To test this, a vaccine expressing cRL1-CTB and mmCT on the chromosome was administered to mice with one booster at day 35 (FIGS. 8A and 8B). However, the combination of mmCT and cRL1-CTB had a minimal effect on the immune response to LPS but greatly attenuated the systemic and mucosal immune response to CTB as compared with the vaccine expressing only mmCT (FIGS. 8C and 8D; FIGS. 11A and 11B).

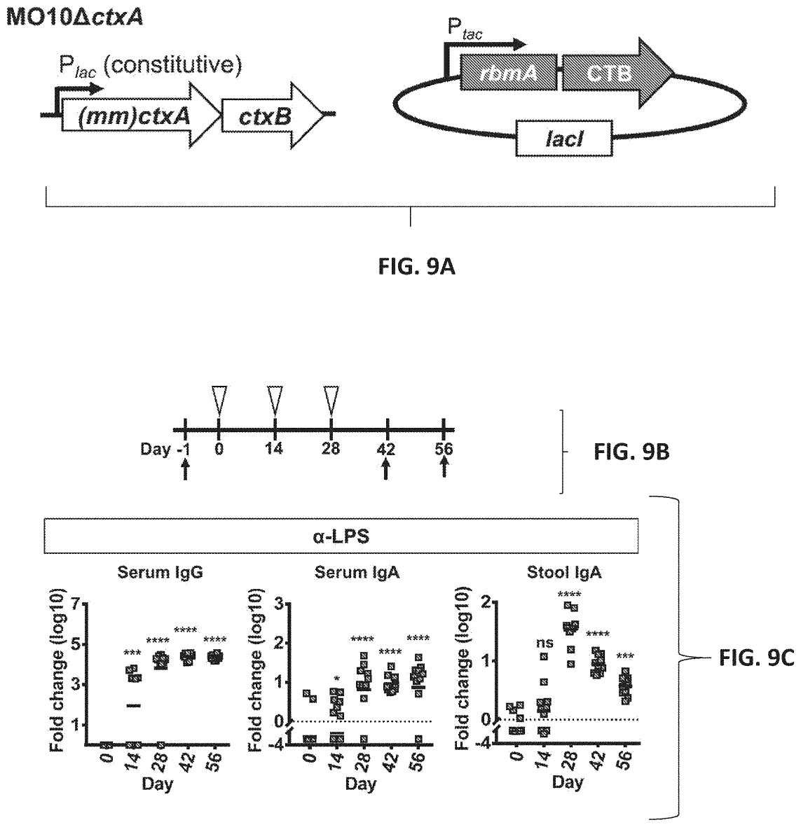

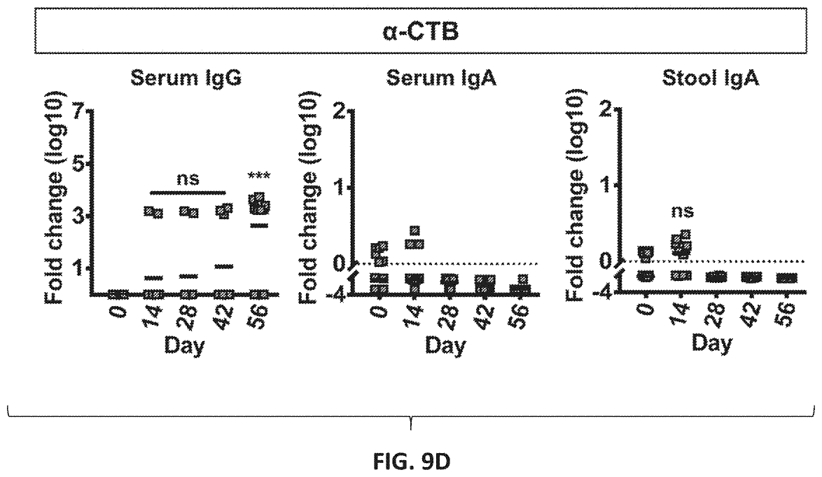

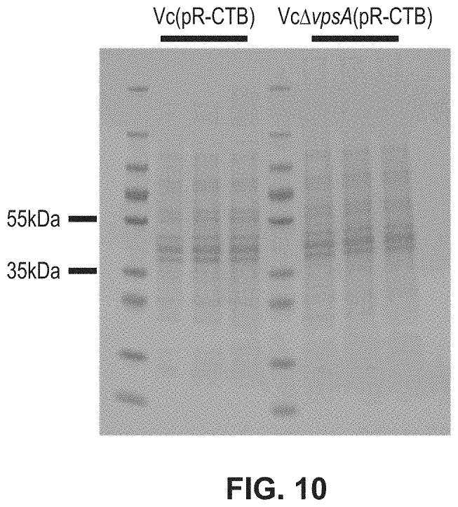

To determine whether this represented a tolerance response, the impact of delivery of greater amounts of CTB was assessed through expression of R-CTB from a plasmid along with constitutive expression of chromosomally-encoded mmCT (FIG. 9A). This vaccine was administered sublingually to mice according to the schedule shown in FIG. 9B with boosters administered on days 14 and 28. The immune response to LPS was not significantly different from other vaccines expressing mmCT (FIGS. 9C and 11A). However, the immune response to CTB was greatly attenuated, indicating that higher doses of CTB increase tolerance specifically to CTB (FIGS. 9D and 11B).

Adjuvanted vaccines that can be easily administered to prevent epidemic and endemic diarrheal disease will greatly improve quality of life in resource-poor settings. The inventors' goal is to develop an antigen presentation platform that does not require purification of antigens or adjuvants. Described herein is the design of a live-attenuated vaccine platform that is capable of synthesizing mmCT, its own mucosal adjuvant, in vivo and can be delivered sublingually. mmCT improved the magnitude and longevity of the mucosal immune response to V. cholerae LPS and elicited both systemic and mucosal responses to CTB. However, inclusion of CTB as an antigen fused to the biofilm matrix protein RbmA dampened the immune response to CTB in a dose-dependent fashion. It was hypothesized that this is an antigen-specific tolerance response that results from the CTB peptide shared by R-CTB and mmCT.

The adjuvanticity of purified mmCT has previously been demonstrated (14, 15). A mechanism is indicated by studies of the dmLT adjuvant, an attenuated variant of the closely related heat-labile toxin of enterotoxigenic E. coli. Purified dmLT was shown to have adjuvant activity comparable to that of native cholera toxin when administered sublingually, likely by promoting proliferation of specific Th1 and Th17 cells (23-25).

Detoxified cholera toxin derivatives have previously been incorporated into live-attenuated V. cholerae strains and delivered via nares (26). The intranasal route of administration circumvents the requirement for passage through the acidic environment of the stomach but also has disadvantages. Despite its documented safety in an oral formulation, intranasal administration of a dmLT-adjuvanted influenza vaccine was correlated with onset of Bell's palsy in healthy adults (27-29). In this study, mmCT was delivered through constitutive synthesis by a live-attenuated vaccine administered via the sublingual route. The safety of mmCT, which was developed as an alternative to dmLT, has not been investigated in humans. Therefore, future work will be necessary to determine the safety of sublingually administered mmCT of the current vaccine formulation.

The sublingual route of administration also has disadvantages. The sublingual space is subject to frequent evacuations through deglutition. To increase antigen residence time, investigators delivered the inactivated polio vaccine combined with the adjuvant dmLT to the sublingual space in a thermo-responsive gel. This formulation elicited mucosal IgA and neutralizing antibodies (30). The data described herein suggest that the live-attenuated vaccine formulation induces a robust systemic and mucosal immune response without the need for depot formulation. V. cholerae has a propensity to attach to diverse surfaces (31, 32). Therefore, one advantage of the live-attenuated vaccine vector described herein compared with the inactivated viral vaccine may be its ability to interact with the sublingual surface. These findings warrant explorations of the interaction between live-attenuated vaccine and the sublingual mucosa and the impact of depot technology on immunogenicity.

Provided herein is a sublingually administered and self-adjuvanting, live-attenuated vaccine vector that induces a robust mucosal IgA response to Vibrio cholerae LPS and cholera toxin. This vaccine does not require additional preparation or protein purification, as the cholera toxin-based adjuvant is constitutively produced during administration in the sublingual space. This has implications for development of future vaccines, especially those based on live-attenuated V. cholerae vector strains. Furthermore, this innovation can simplify manufacturing and administration costs of vaccines effective against diarrheal pathogens.

### Materials and Methods

Bacterial Strains and Culture Conditions.

Vibrio cholerae strains were cultured in Luria-Bertani (LB) broth supplemented with 100 μg/mL streptomycin at 27° C., with shaking at 200 rpm. Escherichia coli was grown in LB broth at 37° C. with shaking. Where necessary, plasmids were maintained with 100 μg/mL of ampicillin in the culture medium. Protein production from plasmid-borne Ptac was induced with 0.5 mM IPTG (β-d-l-thiogalactopyranoside). Frozen stocks were maintained in 15% glycerol at −80° C. Strains used in this study are listed in Table 3.

Table 3: Strains and plasmids used in this study.

TABLE 3
Strains and plasmids used in this study.

| | Description | Reference |
|---|---|---|
| Strains/Plasmid | | |
| V. cholerae | | |
| PW17 | N16961 (El Tor, Inaba); Sm^{r} | (1) |
| PW724 | MO10; Sm^{r} | (2) |
| PW839 | MO10ΔctxA; Sm^{r} | (3) |
| PW1947 | M10ΔctxA, P_{lacz}mmCT; Sm^{r} | This study |
| PW139 (Bengal-2) | MO10ΔattRS1; Sm^{r} | (2) |
| PW1159 | MO10ΔattRS1ΔtcpA; Sm^{r} | This study |
| PW1938 | M10ΔattRS1ΔtcpA, P_{lacz}mmCT; | This study |
| | Sm^{r} | |
| PW1843 | M10ΔattRS1ΔtcpA, PrbmA- | This study |
| | rbmA (R116A, R234A)-ctxB; | |
| | Sm^{r} | |
| PW1850 | M10ΔattRS1ΔtcpA, P_{lacz}-A | This study |
| | mmCT, P_{rbmA}-rbmA(R116A, | |
| | R234A)-ctxB; Sm^{r} | |
| PW1935 | M10ΔattRS1ΔtcpA, P_{rbmA}- | This study |
| | rbmA-eltB-estA(A14H); Sm^{r} | |
| PW1934 | M10ΔattRS1ΔtcpA, P_{lacz}A- | This study |
| | mmCT, P_{rbmA}-rbmA-eltB- | |
| | estA(A14H); Sm^{r} | |
| E. coli | | |
| SM10λpir | thi thr leu tonA lacY supE | (4) |
| | recA::RP4-2-Tc::Mu (λpirR6K); | |
| | Km^{r} | |
| Plasmids | | |
| pWM91 | oriR6K mobRP4 lacI pTac tnp | (5) |
| | mini-Tn10; Km^{r} Ap^{r} | |
| pFLAG-CTC-rbmA-ctxB | IPTG-inducible expression of | (6) |
| | RbmA-CTB fusion protein; Ap^{r} | |
| pWM91-lacZ::mmCT | pWM91 carrying in-frame | This study |
| | mmCT insertion into lacZ for | |
| | homologous recombination; Ap^{r} | |
| pWM91- | pWM91 carrying in-frame | |
| rbmA(R116A, R234A)- | insertion of sequence encoding | |
| ctxB | the indicated R to A mutations | |
| | in RbmA and CTB at the 3′ end | |
| | of rbmA for homologous | |
| | recombination | |
| pWM91-rbmA-eltB-) | pWM91 carrying in-frame | This study |
| estA(A14H | insertion of LTb-Sta_{A14}H fusion | |
| | protein between the 3′ end of | |
| | rbmA and downstream | |
| | sequence; Ap^{r} | |
| pHT3 | pCVD442 carrying unmarked, | (7) |
| | in-frame deletion of tcpA; Ap^{r} | |

DNA Manipulations and Strain Construction.

All oligonucleotides were synthesized by Integrated DNA Technologies and are detailed in Tables 4 and 5. Restriction enzymes were purchased from New England Biolabs (NEB) and used according to manufacturer's instructions. Gibson assembly of DNA fragments was carried out with the NEBuilder HiFi DNA Assembly kit (NEB). PCR reactions were performed with GoTaq polymerase (Promega) for screening and Q5 High-Fidelity polymerase (NEB) for cloning. Genomic DNA from wild-type MO10, a V. cholerae O139 serotype strain, was used as the PCR template unless otherwise noted. Construction of vaccine strain harboring a deletion in tcpA. A sacB-encoding suicide vector carrying a deletion construct for tcpA was introduced into MO10ΔCTXΦΔtcpA by conjugation (33). Homologous recombination and positive deletion clone selection was carried out as described below.

TABLE 4
Primers used for strain construction in this study.

| Primer | Sequence* |
|---|---|
| PlaczI::mmCT | |
| mmCT_1F | GATCATTTGGTAATAGGTATCGATTAAATAAGGAGG |
| mmCT_1R | TGCTTTATTTCGTCGGGCGGGCGACTATC |
| mmCT_2F | CCGCCCGACGAAATAAAGCAGTCAGGTGGTCTTATGC |
| mmCT_2R | ATAACCATCTGCTGCTGGAGCAATATCTAAGTTACTG |
| mmCT_3F | ATTGCTCCAGCAGCAGATGGTTATGGATTGGGCAGGTTTC |
| mmCT_3R | ATCACCCGTGATTGTtccGCTACTATCCCCACAACCCGGCGGTGCATGATG |
| mmCT_4F | GATAGTAGCggaACAATCACGGGTGATACTTGCGATGAAAAAACCCAAAGTC |
| mmCT_4R | GATTGGTATTCGTCagcGAATTTTACACCTAGACTTTG |
| mmCT_5F | GTAAAATTCgctGACGAATACCAATCTAAAGTTAAAAGAC |
| mmCT_5R | GTATTGCACAGGTTARTTTGCCATACTAATTGCG |
| lacZ_1F | GCGCGCGCGAGCTCAAGCCTTACATACAGGCCAGCG |
| lacZ_1R | CGATACCTATTACCAAATGATCACACAAGGGTG |
| lacZ_2F | GCAAATTAACCTGTGCAATACGAAGGGGGC |
| lacZ_2R | GCGCGCGCGAGCTCGCTGGACTTTTTTGACTTCATGTAATG |
| rbmA(R116A, | |
| R234A)-ctxB | |
| rbmA-RR_1F | ATTGGGTACCGGGCCCCCCCGCCTTAGCGCCAGTTGTAAAAAC |
| rbmA-RR_1R | tgcTGTAACGTTCAACATACGACCATCAGTAAGAG |
| rbmA-RR_2F | ATGGTCGTATGTTGAACGTTACAgcaGGTTTC |
| rbmA-RR_2R | GGATAGCTTTTATCAAAATTAATACCtgcCACTTTTG |
| rbmA-RR_3F | gcaGGTATTAATTTTGATAAAAGCTATCCAGCGGGCG |
| rbmA-RR_3R | ATCGATACCGTCGACCTCGAAAAGTCAATATAAAGCCATTATTAGAAC |
| RbmA-eltB- | |
| estA(A14H) | |
| rbmA_1F | ATTGGGTACCGGGCCCCCCCCTCTTACTGATGGTCGTATG |
| rbmA_1R | CCACTGTCATTGACTGTTCC |
| rbmA_2F | GTCGTATGTATAAAAAAACCG |
| rbmA_2R | CTTATCGATACCGTCGACCTCGATAGCATCAATGACCCAAAC |

*Start and stop codons are shown in bold font, sequence for the ribosome binding site is shown in italics. Relevant restriction site sequences are underlined and italicized. Mutations introduced in the sequence encoding mmCT are underlined and specific point mutations are shown in lowercase letters.

TABLE 5
Oligonucleotides synthesized for
strain construction.
Construct^{a}

PlacZ::mmCT
GTATCGATTAAATAAGGAGGAATAAACCATGGTAAAGATAATCTTCGTGT
TCTTCATCTTCCTGAGCAGCTTTTCGTACGCTAACGATGATAAGCTCTAT
CGCGCAGATAGTCGCCCGCCCGACG
rbmA-eltB-estA(A14H)
GAGTGGAACAGTCAATGACAGTGGTAaagaagATGAATAAAGTAAAATGT
TATGTTTTATTTACGGCGTTACTATCCTCTCTATGTGCATACGGAGCTCC
CCAGTCTATTACAGAACTATGTTCGGAATATCGCAACACACAAATATATA
CGATAAATGACAAGATACTATCATATACGGAATCGATGGCAGGCAAAAGA
GAAATGGTTATCATTACATTTAAGAGCGGCGCAACATTTCAGGTCGAAGT
CCCGGGCAGTCAACATATAGACTCCCGCCATTGAAGGATGAAGGACACAT
TAAGAATCACATATCTGACCGAGACCAAAATTGATAAATTATGTGTATGG
AATAATAAAACCCCCAATTCAATTGCGGCAATCAGTATGGAAAACgatcc
ccgggtaccgagctcgAATAGTAGCAATTACTGCTGTGAATTGTGTTGTA
ATCCTcatTGTACCGGGIGCTATTAAATTTACCTAGTCACTTAGTCGTAT
GTATAAAAAACCG
^{a}Start and stop codons are shown in bold type and specific point mutations are shown in lowercase letters. Sequence encoding the linker region between eltB and estA is in bolded lowercase font.

Construction of Vaccine Strain Expressing Plasmid-Encoded R-CTB.

The previously described pFLAG-CTC derivative carrying the gene encoding the B subunit of cholera toxin (CTB) fused to the gene encoding wild-type RbmA (R-CTB) under an IPTG-inducible promoter was introduced into MO10ΔCTXΦΔtcpA by electroporation (34). Protein production in positive transformants was verified by Western blot analysis using an anti-CTB antibody as described below.

Construction of Chromosomal RΔ-CTB Vaccine Strain.

The rbmA-ctxB sequence was amplified from the pFLAG-CTC plasmid carrying R-CTB using the rbmA-RR primer series listed in Table 4. The two point mutations R116A and R234A were introduced by incorporation into the amplification primers. Gibson assembly was used to ligate the fragments into XhoI-linearized suicide plasmid pWM91. Plasmids from positive E. coli clones were isolated and confirmed by sequencing. Homologous recombination into the chromosomal rbmA gene of the MO10MCTXcMtcpA strain and positive clone selection was carried out as described below.

Construction of PlacZmmCT in V. cholerae.

The mmCT sequence was obtained by Gibson assembly of PCR fragments amplified from genomic DNA with the exception of the first 97 bases, which were amplified from an oligonucleotide synthesized to contain the proximal ribosome binding motif from the Ptrc promoter and the first 97 nucleotides of the ctxA gene (Table 5). Mutations of mmCT (15) and stop codons distal to the ribosome binding site were introduced on primers (Table 4) and confirmed in the assembled product by sequencing. This was then ligated between two PCR fragments of the lacZ gene by Gibson assembly to obtain the final construct. This lacZ-mmCT construct was digested using SacI and ligated into the sacB-encoding suicide plasmid pWM91. Plasmids from positive E. coli clones were isolated, and the sequence of the inserted DNA was confirmed by sequencing with M13 primers. Homologous recombination into the MO10MCTXcMtcpA strain and positive clone selection was carried out as previously described (35, 36).

Vaccine Preparation.

Protein induction. Frozen stocks were inoculated into 3 mL of LB supplemented with streptomycin and ampicillin (LB-Sm/Amp) and cultured at 27° C. overnight. For strains carrying plasmid-encoded R-CTB, this starter culture was collected by centrifugation, washed once with LB-Sm/Amp, and sub-cultured into 25 mL of fresh LB-Sm/Amp in a 250-mL flask. After incubating for 6-8 h at 27° C. with shaking, IPTG was added to a final concentration of 0.5 mM, and the culture was incubated for an additional 2 h at 27° C. with shaking.

Trichloroacetic Acid (TCA) Precipitation of Secreted Proteins.

Proteins secreted into the supernatant were precipitated with TCA and washed with acetone. Briefly, spent supernatant was passed through a 0.2 μm filter to remove bacterial cells. TCA was added to the cell-free supernatant to a final concentration of 10% and the supernatant was incubated overnight at 4° C. with gentle mixing. Precipitated proteins were collected by centrifugation and washed three times with ice-cold acetone. Residual acetone was evaporated by brief incubation at 95° C. The protein pellet was resuspended in 4× Laemmli buffer containing β-mercaptoethanol and prepared for Western blot as described below.

Preparation of Whole Cell Vaccines.

After protein induction for strains carrying pR-CTB or after overnight culture for the strains encoding chromosomal RΔ-CTB vaccine constructs, the bacterial culture was centrifuged at 5,000×g for 15 min at 4° C. to collect cells. The supernatant was passed through a 0.2 μm filter, and the resulting cell-free supernatant was used for TCA precipitation and Western blot analysis. The remaining bacterial pellet was washed three times with 12 mL of sterile PBS and finally resuspended in 1 mL of PBS. This constituted the live-attenuated whole cell vaccine. For each vaccine preparation, 10 μL was removed to quantify colony forming units (CFU), and 20 μL was reserved for Western blot analysis. For each immunization, vaccines expressing mmCT were kept on ice for less than 1 h before administration, and all vaccines were used within 2 h of preparation. Each vaccine dose consisted of 108-109 cells in 10 μL.

Western Blot Analysis.

Supernatants and cell pellet samples were separated by centrifugation. TCA precipitation of the supernatant was performed prior to detection of mmCT. These samples were combined with 4× Laemmli buffer containing β-mercaptoethanol, sonicated in an ice bath, boiled for 5 min, and briefly centrifuged to remove particulates. Proteins were resolved on a denaturing 4-20% gradient Tris-HCl gel and then transferred onto a polyvinylidene difluoride membrane by semi-dry transfer (BioRad). The membrane was blocked in tris-buffered saline with 0.1% Tween (TBS-T) and 5% skim milk for 2 h at room temperature with gentle shaking. Fresh blocking solution containing primary antibody was added in a 1:1,000 dilution. Rabbit-derived serum raised against both the A and B subunits of cholera toxin (Sigma) was used to detect mmCT. After overnight incubation with primary antibodies, the membrane was washed 3 times with TBS-T. Membranes were then incubated for 2 h at room temperature with 1:5,000 diluted HRP-conjugated anti-rabbit secondary antibody (Cell Signaling) and developed using an ECL Western blotting substrate (Pierce).

Quantification of R-CTB by Densitometry.

Known concentrations of purified CTB (List Laboratories) were resolved by SDS-PAGE alongside RbmA-CTB samples (plasmid and chromosomal) and used as standards for quantification. ImageJ was used to generate a standard curve fitted to the intensities of bands corresponding to the CTB standards. Concentration of RbmA-CTB was calculated using the linear portion of the standard curve.

Quantification of mmCT in Supernatants by ELISA.

The amount of mmCT secreted into the supernatant by the live vaccine suspension was quantified by GM1 ELISA as previously described (37). The supernatant of a wild-type strain that does not express mmCT was similarly prepared and used as a negative control. One hundred ng of bovine monosialoganglioside GM1 (Sigma) in sodium carbonate buffer was added to each well of a 96 well microtiter plate (Nunc, Maxisorp), incubated overnight at room temperature, and then washed in PBS-T. Serial dilutions of cell-free supernatants in PBS were applied to GM1-coated wells. Purified CTB was prepared in a 1 μg/mL concentration in PBS, and serial dilutions were also applied to GM1-coated wells to generate a standard curve. The plates were incubated overnight at room temperature, washed in PBS-T, and blocked in PBS-BSA. Monoclonal anti-CTB IgG (Fisher, PIMA183519) diluted to 1 μg/mL in PBS-T-BSA was added to the plates and incubated overnight. The plates were probed with HRP-conjugated goat anti-mouse antibodies (Bethyl Laboratories) and developed as described above.

### Immunization and Sample Collection

Animals.

Female, 6-8-week old BALB/c mice were used in all immunization experiments. Mice were purchased from Charles River Laboratories and housed in a biosafety level two facility at Boston Children's Hospital with food and water ad libitum. Mice were acclimatized for 5 days. All procedures had been previously approved by the Institutional Animal Care and Use Committee.

Sublingual Administration of Live-Attenuated Vaccine.

Mice were anesthetized by intraperitoneal injection with a cocktail of ketamine (100 mg/kg) and xylazine (10 mg/kg), then held upright while 10 μL of the vaccine was delivered under the tongue by a micropipette directed toward the floor of the mouth. Mice were maintained in the upright position for 2 min before resting, ventral side down, for at least 30 min until regaining consciousness.

Collection of Blood and Stool Samples.

Blood and stool samples were collected one day before vaccination and at the designated time points throughout the study period. Fresh stool pellets were frozen at −80° C. until use. Blood was collected from the tail vein using capillary tubes with clot activator (Sarstedt). Sera were obtained by clearing the clotted blood with centrifugation at 10,000×g for 5 min at room temperature and stored at −20° C. Stool samples were prepared as previously described (38). Briefly, pellets were thawed on ice, transferred to 15-mL conical tubes containing 3 mL of chilled resuspension solution (0.1 mg/mL soybean trypsin inhibitor, 3:1 mixture of PBS to 0.1 M EDTA), thoroughly homogenized, and centrifuged at 650×g for 10 min at room temperature. The supernatant was collected and centrifuged once more at 15,300×g for 10 min at 4° C. Phenylmethane sulfonyl fluoride (PMSF) was added to the supernatant to a final concentration of 2 mM. Stool samples were kept at −20° C. or at −80° C. for long term storage.

### Enzyme-Linked Immunosorbent Assays

Quantification of CTB-Specific Antibodies by ELISA.

Standard Curve:

CTB-specific IgA was not available for use in a standard curve. Therefore, to assess linearity, standard curves were generated by capturing IgA and IgG in reference mouse serum with goat anti-mouse IgG or IgA. Microtiter plate wells were incubated overnight with 100 μg of goat anti-mouse IgG or IgA diluted in sodium carbonate buffer. The wells were washed in PBS-T and blocked with PBS-BSA. Reference mouse serum (Bethyl Laboratory) was diluted to 1 μg/mL of total IgG or IgA and applied to the wells. The wells were washed after overnight incubation, then probed with HRP-conjugated goat anti-mouse antibodies and developed as described above for quantification of mmCT.

Test Samples:

Microtiter plates were coated with GM1 followed by purified CTB as described above. The plates were blocked in PBS-BSA and washed in PBS-T. Serially diluted sera or stool samples were applied to the wells and incubated overnight. Serum dilutions ranged from 1:50 to 1:6400, and stool dilutions ranged from 1:2 to 1:128. The plates were probed and developed as described above.

Quantification of Total Stool IgA by ELISA.

Total fecal IgA was used to normalize antigen-specific IgA in the stool. Each well of a microtiter plates was coated with 100 ng of goat anti-mouse IgA antibody in sodium bicarbonate buffer and incubated overnight. Plates were washed in PBS-T and blocked in PBS-BSA. Stool samples were serially diluted from 1:200 to 1:25,600 in PBS-T-BSA and added to the plates. The plates were incubated overnight and then probed and developed as described above. Standard curves were generated as described for CTB/LTB-specific antibodies.

Lipopolysaccharide (LPS) Extraction and Measurement of O-Antigen Specific Antibodies.

LPS was extracted from 50 mL of a V. cholerae MO10 (serotype O139) overnight culture using a commercial kit (Bulldog Bio). Serum and stool antibodies recognizing the O1 or O139 serotypes were quantified As previously described (39). A 1:1,000 dilution of LPS in sodium carbonate buffer was applied to microtiter plates and incubated overnight. The plates were washed in PBS-T and blocked for 40 min at 37° C. in PBS-BSA. Serum and stool samples were applied to the plates in dilutions similar to those used to measure CTB-specific antibodies. The plates were incubated for 90 min at 37° C. and then washed in PBS-T. Plates were incubated for 90 min at 37° C. after addition of 100 ng of HRP-conjugated goat anti-mouse antibodies per well. Plates were developed using the same protocol described for quantification of CTB-specific antibodies.

Statistical analysis. Statistical analyses were performed in GraphPad Prism 7. One-way ordinary ANOVA with Dunnett's test was used for multiple comparisons. Two-tailed, un-paired Student's t test was used for pair-wise comparisons. All vaccine groups consisted of ten mice. Error bars indicate standard deviations unless otherwise noted. Western blot and photographic images are representative of experimental triplicates.

### REFERENCES

1. Qadri F, Islam T, Clemens J D. 2017. Cholera in Yemen—An Old Foe Rearing Its Ugly Head. N Engl J Med 377:2005-2007.
2. Zarocostas J. 2017. Cholera outbreak in Haiti—from 2010 to today. Lancet 389:2274-2275.
3. Brown V, Reilley B, Ferrir M C, Gabaldon J, Manoncourt S. 1997. Cholera outbreak during massive influx of Rwandan returnees in November, 1996. Lancet 349:212.
4. Peterson K M, Gellings P S. 2018. Multiple intraintestinal signals coordinate the regulation of Vibrio cholerae virulence determinants. Pathog Dis 76.
5. Mosley J F, 2nd, Smith L L, Brantley P, Locke D, Como M. 2017. Vaxchora: The First FDA-Approved Cholera Vaccination in the United States. P T 42:638-640.
6. Ilboudo P G, Le Gargasson J B. 2017. Delivery cost analysis of a reactive mass cholerA vaccination campaign: a case study of Shanchol vaccine use in Lake Chilwa, Malawi. BMC Infect Dis 17:779.
7. Azman A S, Parker L A, Rumunu J, Tadesse F, Grandesso F, Deng L L, Lino R L, Bior B K, Lasuba M, Page A L, Ontweka L, Llosa A E, Cohuet S, Pezzoli L, Sodjinou D V, Abubakar A, Debes A K, Mpairwe A M, Wamala J F, Jamet C, Lessler J, Sack D A, Quilici M L, Ciglenecki I, Luquero F J. 2016. Effectiveness of one dose of oral cholera vaccine in response to an outbreak: a case-cohort study. Lancet Glob Health 4:e856-e863.
8. Poncin M, Zulu G, Voute C, Ferreras E, Muleya C M, Malama K, Pezzoli L, Mufunda J, Robert H, Uzzeni F, Luquero F J, Chizema E, Ciglenecki I. 2018. Implementation research: reactive mass vaccination with single-dose oral cholera vaccine, Zambia. Bull World Health Organ 96:86-93.
9. Naficy A, Rao M R, Paquet C, Antona D, Sorkin A, Clemens J D. 1998. Treatment and vaccination strategies to control cholera in sub-Saharan refugee settings: a cost-effectiveness analysis. JAMA 279:521-525.
10. Kim S Y, Choi Y, Mason P R, Rusakaniko S, Goldie S J. 2011. Potential impact of reactive vaccination in controlling cholera outbreaks: an exploratory analysis using a Zimbabwean experience. S Afr Med J 101:659-664.
11. Blaauboer S M, Mansouri S, Tucker H R, Wang H L, Gabrielle V D, Jin L. 2015. The mucosal adjuvant cyclic di-GMP enhances antigen uptake and selectively activates pinocytosis-efficient cells in vivo. Elife 4.
12. Fukasaka M, Asari D, Kiyotoh E, Okazaki A, Gomi Y, Tanimoto T, Takeuchi O, Akira S, Hori M. 2015. A Lipopolysaccharide from Pantoea agglomerans Is a Promising Adjuvant for Sublingual Vaccines to Induce Systemic and Mucosal Immune Responses in Mice via TLR4 Pathway. PLoS ONE 10:e0126849.
13. Lee C H, Masso-Welch P, Hajishengallis G, Connell T D. 2011. TLR2-dependent modulation of dendritic cells by LT-IIa-B5, a novel mucosal adjuvant derived from a type II heat-labile enterotoxin. J Leukoc Biol 90:911-921.
14. Larena M, Holmgren J, Lebens M, Terrinoni M, Lundgren A. 2015. Cholera toxin, and the related nontoxic adjuvants mmCT and dmLT, promote human Th17 responses via cyclic AMP-protein kinase A and inflammasome-dependent IL-1 signaling. The Journal of Immunology 194:3829-3839.
15. Lebens M, Terrinoni M, Karlsson S L, Larena M, Gustafsson-Hedberg T, Kallgard S, Nygren E, Holmgren J. 2016. Construction and preclinical evaluation of mmCT, a novel mutant cholera toxin adjuvant that can be efficiently produced in genetically manipulated Vibrio cholerae. Vaccine 34:2121-2128.
16. Hartman A B, Van De Verg L L, Venkatesan M M. 1999. Native and mutant forms of cholera toxin and heat-labile enterotoxin effectively enhance protective efficacy of live attenuated and heat-killed Shigella vaccines. Infect Immun 67:5841-5847.
17. Kang J O, Lee J B, Chang J. 2016. Cholera Toxin Promotes Th17 Cell Differentiation by Modulating Expression of Polarizing Cytokines and the Antigen-Presenting Potential of Dendritic Cells. PLoS ONE 11:e0157015.
18. Datta S K, Sabet M, Nguyen K P, Valdez P A, Gonzalez-Navajas J M, Islam S, Mihajlov I, Fierer J, Insel P A, Webster N J, Guiney D G, Raz E. 2010. Mucosal adjuvant activity of cholera toxin requires Th17 cells and protects against inhalation anthrax. Proc Natl Acad Sci USA 107:10638-10643.
19. Hirota K, Turner J E, Villa M, Duarte J H, Demengeot J, Steinmetz O M, Stockinger B. 2013. Plasticity of Th17 cells in Peyer's patches is responsible for the induction of T cell-dependent IgA responses. Nat Immunol 14:372-379.
20. Liao J, Smith D R, Brynjarsdottir J, Watnick P I. 2018. A self-assembling whole cell vaccine antigen presentation platform. J Bacteriol in press.
21. Waldor M K, Mekalanos J J. 1994. Emergence of a new cholera pandemic: molecular analysis of virulence determinants in Vibrio cholerae O139 and development of a live vaccine prototype. The Journal of Infectious Diseases 170:278-283.
22. Giglio K M, Fong J C, Yildiz F H, Sondermann H. 2013. Structural basis for biofilm formation via the Vibrio cholerae matrix protein RbmA. J Bacteriol 195:3277-3286.
23. Sjokvist Ottsjo L, Flach C F, Clements J, Holmgren J, Raghavan S. 2013. A double mutant heat-labile toxin from Escherichia coli, LT(R192G/L211A), is an effective mucosal adjuvant for vaccination against Helicobacter pylori infection. Infect Immun 81:1532-1540.
24. Luo Q, Vickers T J, Fleckenstein J M. 2016. Immunogenicity and Protective Efficacy against Enterotoxigenic Escherichia coli Colonization following Intradermal, Sublingual, or Oral Vaccination with EtpA Adhesin. Clin Vaccine Immunol 23:628-637.
25. Raghavan S, Ostberg A K, Flach C F, Ekman A, Blomquist M, Czerkinsky C, Holmgren J. 2010. Sublingual immunization protects against Helicobacter pylori infection and induces T and B cell responses in the stomach. Infect Immun 78:4251-4260.
26. Fontana M R, Monaci E, Yanqing L, Guoming Q, Duan G, Rappuoli R, Pizza M. 2000. IEM101, a naturally attenuated Vibrio cholerae strain as carrier for genetically detoxified derivatives of cholera toxin. Vaccine 19:75-85.
27. El-Kamary S S, Cohen M B, Bourgeois A L, Van De Verg L, Bauers N, Reymann M, Pasetti M F, Chen W H. 2013. Safety and immunogenicity of a single oral dose of recombinant double mutant heat-labile toxin derived from enterotoxigenic Escherichia coli. Clin Vaccine Immunol 20:1764-1770.
28. Lundgren A, Bourgeois L, Carlin N, Clements J, Gustafsson B, Hartford M, Holmgren J, Petzold M, Walker R, Svennerholm A M. 2014. Safety and immunogenicity of an improved oral inactivated multivalent enterotoxigenic Escherichia coli (ETEC) vaccine administered alone and together with dmLT adjuvant in a double-blind, randomized, placebo-controlled Phase I study. Vaccine 32:7077-7084.
29. Lewis D J, Huo Z, Barnett S, Kromann I, Giemza R, Galiza E, Woodrow M, Thierry-Carstensen B, Andersen P, Novicki D, Del Giudice G, Rappuoli R. 2009. Transient facial nerve paralysis (Bell's palsy) following intranasal delivery of a genetically detoxified mutant of Escherichia coli heat labile toxin. PLoS ONE 4:e6999.
30. White J A, Blum J S, Hosken N A, Marshak J O, Duncan L, Zhu C, Norton E B, Clements J D, Koelle D M, Chen D, Weldon W C, Oberste M S, Lal M. 2014. Serum and mucosal antibody responses to inactivated polio vaccine after sublingual immunization using a thermoresponsive gel delivery system. Hum Vaccin Immunother 10:3611-3621.
31. Wong E, Vaaje-Kolstad G, Ghosh A, Hurtado-Guerrero R, Konarev P V, Ibrahim A F, Svergun D I, Eijsink V G, Chatterjee N S, van Aalten D M. 2012. The Vibrio cholerae colonization factor GbpA possesses a modular structure that governs binding to different host surfaces. PLoS Pathog 8:e1002373.
32. Benktander J, Angstrom J, Karlsson H, Teymournejad O, Linden S, Lebens M, Teneberg S. 2013. The repertoire of glycosphingolipids recognized by Vibrio cholerae. PLoS ONE 8:e53999.
33. Thelin K H, Taylor R K. 1996. Toxin-Coregulated Pilus, but Not Mannose-Sensitive Hemagglutinin, Is Required for Colonization by Vibrio cholerae O1 El Tor Biotype and O139 Strains. Infection and Immunity 64:2853-2856.
34. Absalon C, Ymele-Leki P, Watnick P I. 2012. The bacterial biofilm matrix as a platform for protein delivery. mBio 3:e00127-00112-e00127-00112.
35. Butterton J R, Beattie D T, Gardel C L, Carroll P A, Hyman T, Killeen K P, Mekalanos J J, Calderwood S B. 1995. Heterologous antigen expression in Vibrio cholerae vector strains. Infect Immun 63:2689-2696.
36. Metcalf W W, Jiang W, Daniels L L, Kim S K, Haldimann A, Wanner B L. 1996. Conditionally replicative and conjugative plasmids carrying lacZ alpha for cloning, mutagenesis, and allele replacement in bacteria. Plasmid 35:1-13.
37. Ryan E T, Butterton J R, Smith R N, Carroll P A, Crean T I, Calderwood S B. 1997. Protective Immunity against Clostridium difficile Toxin A Induced by Oral Immunization with a Live, Attenuated Vibrio cholerae Vector Strain. Infection and Immunity 65:2941-2949.
38. Ryan E T, Butterton J R, Zhang T, Baker M A, Stanley S L, Jr., Calderwood S B. 1997. Oral immunization with attenuated vaccine strains of Vibrio cholerae expressing a dodecapeptide repeat of the serine-rich Entamoeba histolytica protein fused to the cholera toxin B subunit induces systemic and mucosal antiamebic and anti-V. cholerae antibody responses in mice. Infect Immun 65:3118-3125.
39. Rollenhagen J E, Kalsy A, Saksena R, Sheikh A, Alam M M, Qadri F, Calderwood S B, Kovac P, Ryan E T. 2009. Transcutaneous immunization with a synthetic hexasaccharide-protein conjugate induces anti-Vibrio cholerae lipopolysaccharide responses in mice. Vaccine 27:4917-4922.

1. A composition comprising a decorated Vibrio cholerae bacterium having a fusion protein associated with its extracellular surface, wherein the fusion protein comprises a moiety that binds Vibrio polysaccharide (VPS) and an antigenic moiety.

2. The composition of claim 1, wherein the Vibrio cholerae bacterium is inactivated or live-attenuated.

3. The composition of claim 1, wherein the immunogenicity of the decorated bacterium is increased by at least 10% compared to the same bacterium in the absence of the fusion protein.

4. The composition of claim 1, wherein the fusion protein is non-covalently attached to the surface of the bacterium.

5. The composition of claim 1, wherein the moiety that binds VPS is a lectin.

6. The composition of claim 5, wherein the lectin is RbmA or a C-terminal portion thereof.

7. The composition of claim 1, wherein the antigenic moiety comprises B subunit of cholera toxin (CTB), a Shigella antigen or a heat-stable toxoid from enterotoxic E. coli.

8. The composition of claim 1, wherein the fusion protein is expressed from a plasmid by the bacterium.

9. The composition of claim 1, wherein expression of the fusion protein is under the control of an inducible promoter.

10. The composition of claim 1, wherein expression of the fusion protein is under the control of a chromosomal promoter.

11. The composition of claim 8, wherein at least one additional antigen is chromosomally expressed.

12. The composition of claim 11, wherein the at least one additional antigen comprises multiply-mutated cholera toxin (mmCT).

13. The composition of claim 1, wherein the composition is formulated as a whole cell vaccine.

14. The composition of claim 1, wherein the composition is formulated for sublingual delivery.

15. The composition of claim 1, further comprising an adjuvant.

16. The composition of claim 17, wherein the adjuvant is mmCT.

17. A method for inducing immunity to Vibrio cholerae in a subject, the method comprising: administering at least one dose of the composition of claim 1 to a subject at risk of being exposed to and/or developing cholera, thereby inducing immunity to Vibrio cholerae in the subject.

18. The method of claim 19, wherein the dose is a single dose comprising an effective amount of the composition.

19. The method of claim 20, wherein the dose is administered at least twice.

20. The method of claim 19, wherein the administering does not require sodium bicarbonate in potable water.
